(19) United States
(12) Patent Application Publication
(10) Pub. No.: US 2015/0094759 A1
(43) Pub. Date: Apr. 2, 2015

(54) VASCULATURE CLOSURE DEVICES AND METHODS

(71) Applicant: E-Pacing, Inc., Wilmington, DE (US)

(72) Inventors: Lone Wolinsky, Ramat Gan (IL); Abraham Penner, Tel Aviv (IL); Alon Ben-Yosef, Ramot Manasha (IL)

(21) Appl. No.: 14/566,805

(22) Filed: Dec. 11, 2014

Related U.S. Application Data
(63) Continuation of application No. PCT/US2013/045100, filed on Jun. 11, 2013.
(60) Provisional application No. 61/658,185, filed on Jun. 11, 2012.
(60) Provisional application No. 61/668,868, filed on Jul. 6, 2012.

(51) Int. Cl. A61B 17/00

(52) U.S. Cl. CPC A61B 17/0057; A61B 2017/00592; A61B 2017/00597; A61B 2017/00615; A61B 2017/00623; A61B 2017/0065; A61B 2017/00654; A61B 2017/00659; A61B 2017/00668; A61B 2017/00884; A61B 2017/00893; A61B 2017/0417
USPC 606/213

(57) ABSTRACT

Vasculature closure devices, and systems and methods for their use, are provided. In one embodiment, the vasculature closure device includes an expandable support frame deployable within a vessel, a sealing membrane at least partially supported by the support frame, and a cross-member support extending across at least a portion of the sealing membrane. Upon expanding the support frame, the device is configured to intraluminally position the sealing membrane against a puncture site existing in a wall of the vessel. The cross-member support includes a flexible wire coupled to the sealing membrane at an intermediate portion of the flexible wire and configured to maintain the sealing membrane against the puncture site.

### CROSS-REFERENCE TO RELATED APPLICATIONS

This application is a continuation of PCT Application No. PCT/US2013/045100, filed on Jun. 11, 2013, which claims the benefit of U.S. Provisional Application No. 61/658,185, filed on Jun. 11, 2012, and U.S. Provisional Application No. 61/668,868, filed on Jul. 6, 2012, each of which is incorporated herein by reference in its entirety.

### BACKGROUND

This disclosure relates generally to the field of implantable medical devices and associated methods, and more particularly to devices and methods for closing openings in vessel walls.

During certain endovascular surgery procedures, intravascular catheters are inserted through an incision in the patient's skin and underlying tissue to access an artery or vein. After the surgical procedure is completed and the catheter is removed from the vessel, the puncture providing the access through the patient's vessel wall must be closed. This is quite difficult, not only because of the high blood pressure within an artery, but also because of the many layers of tissue that must be penetrated to reach the vessel to achieve closure.

Physicians currently use a number of methods to close a vessel puncture, which include applying localized compression, sutures, collagen plugs, adhesives, gels, and/or foams. To provide localized compression, the physician applies pressure against the vessel to facilitate natural clotting of the vessel puncture. However, this method can take up to a half hour or more and requires the patient to remain immobilized while providing the compression and to remain in the hospital for a period thereafter for observation. The amount of time necessary to apply compression can, in some circumstances, be even greater, depending upon the levels of anti-clotting agents (e.g., heparin, glycoprotein IIb/IIA antagonists, etc.) administered during the endovascular procedure. In addition, applying localized compression can increase the potential for blood clots at the puncture site to become dislodged. Closing procedures in which sutures, collagen plugs, adhesives, gels, and/or foams are applied suffer from variability and unpredictability associated with implantation procedures, many of which are complicated and require highly technical implantation techniques. Some of these closure methods occasionally cause undesirable deformation of the vessels. Moreover, for newer endovascular procedures, such as abdominal or thoracic aortic aneurysm repair, percutaneous valve replacement and repair, or cardiac ablation, which use large diameter delivery systems typically in the range of 8-25 Fr, these conventional closure methods are suboptimal.

Certain devices and methods have been developed for closing openings in vessel walls. For example, U.S. Patent Application Publication No. 2011/0087270 to Penner et al. provides various examples of vasculature closure devices and methods for deploying and performing treatment using the same.

There remains a need for improved and/or more robust designs and methods for vasculature closure devices and delivery tools, for example, which provide the physician with enhanced reliability, ease and efficiency of delivery of the closure device into its correct location in the vessel, while further minimizing or avoiding the risk of damage to the closure device or to the vessel.

### BRIEF SUMMARY

Vasculature closure devices and systems and methods for their use are provided. According to one aspect, a vasculature closure device is provided. In one embodiment, the vasculature closure device includes an expandable support frame deployable within a vessel, a sealing membrane at least partially supported by the support frame, and a cross-member support extending across at least a portion of the sealing membrane. Upon expanding the support frame, the device is configured to intraluminally position the sealing membrane against a puncture site existing in a wall of the vessel. The cross-member support includes a flexible wire coupled to the sealing membrane at an intermediate portion of the flexible wire and configured to maintain the sealing membrane against the puncture site.

In another embodiment, the vasculature closure device includes an expandable tube deployable within a vessel, and a tether attached to an outer surface of the tube. The tube includes a solid sidewall and is configured to expand from a collapsed configuration into an expanded configuration to intraluminally position the outer surface of the tube against a puncture site existing in a wall of the vessel. The tube also is configured to be flattened into the collapsed configuration.

In a further embodiment, the vasculature closure device includes an expandable support frame deployable within a vessel, a sealing membrane at least partially supported by the support frame, a tether extending away from the sealing membrane, and a securing element coupled to the tether. Upon expanding the support frame, the device is configured to intraluminally position the sealing membrane against a puncture site existing in a wall of the vessel. The securing element is configured to engage an access channel formed in a tissue adjacent the puncture site to prevent intraluminal migration of the device.

In yet another embodiment, the vasculature closure device includes an expandable support frame deployable within a vessel, a sealing membrane at least partially supported by the support frame, and a fixation element extending from the support frame. Upon expanding the support frame, the device is configured to intraluminally position the sealing membrane against a puncture site existing in a wall of the vessel. The fixation element is configured to penetrate the wall of the vessel adjacent the puncture site to prevent intraluminal migration of the device.

According to another aspect, a system for closing a puncture site in a wall of a vessel is provided. In one embodiment, the system includes a vasculature closure device including an expandable support frame deployable within the vessel, and a sealing membrane at least partially supported by the support frame. The vasculature closure device is configured to expand from a collapsed configuration into an expanded configuration to intraluminally position the sealing membrane against the puncture site. The system also includes a containment mechanism including a loop element encircling the vasculature closure device, and an implant holder coupled to the loop element and extending along a longitudinal axis of the vasculature closure device, the implant holder including a proximal element extending beyond a proximal end of the vasculature closure device. The containment mechanism is configured to releasably retain the vasculature closure device in the collapsed configuration and position the vasculature closure device within the vessel, and the proximal element is configured to lead the vasculature closure device toward a proximal portion of the vessel.

In another embodiment, the system includes a vasculature closure device including an expandable support frame deployable within the vessel, and a sealing membrane at least partially supported by the support frame. The vasculature closure device is configured to expand from a collapsed configuration into an expanded configuration to intraluminally position the sealing membrane against the puncture site. The system also includes an inserting tool configured to intraluminally center the vasculature closure device across the puncture site.

In a further embodiment, the system includes a vasculature closure device including an expandable support frame deployable within the vessel, and a sealing membrane at least partially supported by the support frame. The vasculature closure device is configured to expand from a collapsed configuration into an expanded configuration to intraluminally position the sealing membrane against the puncture site. The system also includes an inserting tool configured to deliver therapeutic agents to the puncture site.

According to yet another aspect, a method for closing a puncture site in a wall of a vessel is provided. In one embodiment, the method includes deploying, via a sheath, a vasculature closure device including an expandable support frame and a sealing membrane into the vessel through the puncture site, wherein the support frame is in a collapsed configuration during deployment. The method also includes expanding the support frame into an expanded configuration within the vessel, and positioning the support frame within the vessel to position the sealing membrane against the puncture site to at least partially seal the puncture site. The method further includes engaging a tissue adjacent the puncture site with a securing element of the vasculature closure device to prevent intraluminal migration of the vasculature closure device.

In another embodiment, the method includes deploying, via a sheath, a vasculature closure device including an expandable support frame and a sealing membrane into the vessel through the puncture site, wherein the support frame is in a collapsed configuration during deployment. The method also includes expanding the support frame into an expanded configuration within the vessel, and positioning the support frame within the vessel to position the sealing membrane against the puncture site to at least partially seal the puncture site. The method further includes penetrating the wall of the vessel adjacent the puncture site with a fixation element of the vasculature closure device to prevent intraluminal migration of the vasculature closure device.

In a further embodiment, the method includes deploying, via a sheath, a vasculature closure device including an expandable support frame and a sealing membrane into the vessel through the puncture site, wherein the support frame is retained in a collapsed configuration by a containment mechanism during deployment. The method also includes leading, via a proximal element of an implant holder, the vasculature closure device toward a proximal portion of the vessel, wherein the proximal element extends beyond a proximal end of the vasculature closure device. The method further includes expanding the support frame into an expanded configuration within the vessel to position the sealing membrane against the puncture site to at least partially seal the puncture site.

In yet another embodiment, the method includes deploying, via a sheath, a vasculature closure device including an expandable support frame and a sealing membrane into the vessel through the puncture site, wherein the support frame is in a collapsed configuration during deployment. The method also includes expanding the support frame into an expanded configuration within the vessel. The method further includes pressing a distal tip of an inserting tool against the wall of the vessel, and pulling a tether of the vasculature closure device through a lumen of the inserting tool to center the vasculature closure device across the puncture site.

In yet a further embodiment, the method includes deploying, via a sheath, a vasculature closure device including an expandable support frame and a sealing membrane into the vessel through the puncture site, wherein the support frame is retained in a collapsed configuration during deployment. The method also includes expanding the support frame into an expanded configuration within the vessel to position the sealing membrane against the puncture site to at least partially seal the puncture site. The method further includes delivering a therapeutic agent through a lumen of an inserting tool to the puncture site, an access channel formed in a tissue adjacent the puncture site, or an interface between the vasculature closure device and the vessel.

### BRIEF DESCRIPTION OF THE DRAWINGS

FIG. 1 is an illustration of a prior art vasculature closure device (VCD) implanted intraluminally within a vessel according to one embodiment.

FIGS. 2A and 2B are illustrations of VCDs according to some representative embodiments.

FIGS. 3A-3F are illustrations of VCDs according to some representative embodiments.

FIGS. 4A-4E are partial cross-sectional views illustrating a delivery system and stages of delivering and securing a VCD within a vessel according to one representative embodiment. FIGS. 4F-4I are illustrations of securing elements according to some representative embodiments. FIGS. 4J and 4K are partial cross-sectional views illustrating a delivery system and stages of delivering and securing a VCD within a vessel according to one representative embodiment. FIGS. 4L and 4M are illustrations of securing elements of a VCD according to some representative embodiments. FIG. 4N is a partial cross-sectional view illustrating a delivery system and one stage of delivering and securing a VCD within a vessel according to one representative embodiment.

FIGS. 5A-5C are partial cross-sectional views illustrating a VCD secured within a vessel according to some representative embodiments. FIGS. 5D-5J are illustrations of fixation elements of a VCD according to some representative embodiments.

FIG. 6A is a graph of VCD strain as a function of VCD diameter according to one representative embodiment. FIG. 6B is a graph of strain ratio as a function of VCD free diameter according to one representative embodiment.

FIG. 7A is an illustration of a VCD and corresponding containment mechanism according to one representative embodiment. FIG. 7B is an illustration of an implant holder of a containment mechanism according to one representative embodiment. FIG. 7C is an illustration of a VCD and corresponding containment mechanism according to one representative embodiment. FIG. 7D-7F are illustrations of implant holders of containment mechanisms according to some representative embodiments.

FIGS. 8A and 8B are partial cross-sectional views illustrating an inserting tool and stages of positioning a VCD within a vessel according to one representative embodiment. FIGS. 8C-8G are illustrations of distal tips of inserting tools according to some representative embodiments. FIGS. 8H and 8I are partial cross-sectional views illustrating an inserting tool and stages of securing a VCD within a vessel according to one representative embodiment. FIG. 8J is a cross-sectional view illustrating an inserting tool according to one representative embodiment.

FIG. 9 is a cross-sectional view illustrating an inserting tool according to one representative embodiment.

### DETAILED DESCRIPTION

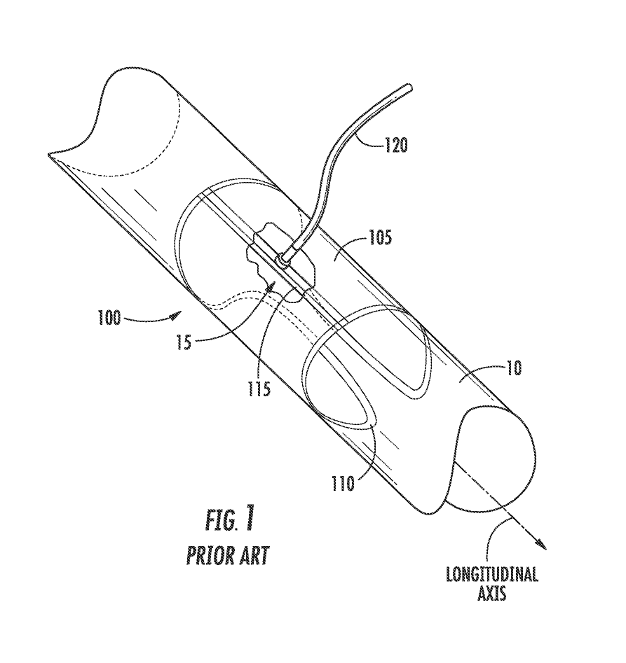

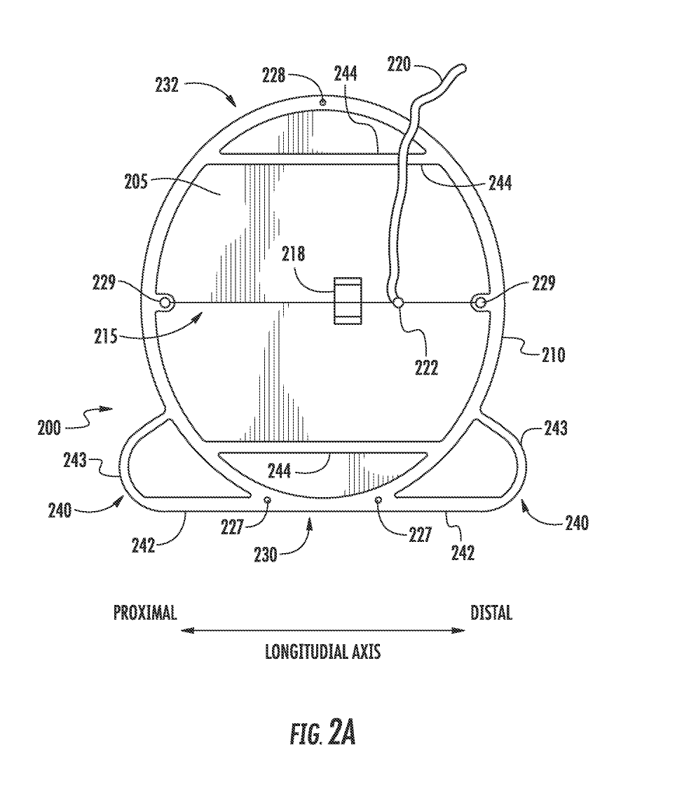

FIG. 1 illustrates an embodiment of a prior art vasculature closure device (VCD) 100 implanted intraluminally within a vessel to facilitate hemostasis and closure of a vessel puncture. Described herein are improved systems and methods for delivering a VCD into a patient in need thereof, which may include and/or be used with the VCD 100 of FIG. 1. Also described herein are improved vasculature closure devices, which may be included in and/or be used with the improved systems and methods for delivery.

A VCD, according to various embodiments described herein, includes at least one sealing membrane and at least one support frame attached to, integrated with, or otherwise supporting the sealing membrane. The support frame is utilized to expand the sealing membrane from a collapsed configuration to an expanded configuration when deployed within a vessel. The support frame can be configured such that it expands enough to force the sealing membrane to move into a position against a vessel puncture. The pressure exerted by the support frame can vary, but is effective to at least partially maintain the VCD at the desired position within the vessel—which at least partially presses the sealing membrane against the vessel puncture. Upon positioning and exerting pressure by the sealing membrane against the vessel puncture, blood leakage is prevented and/or reduced, and hemostasis and healing are promoted. In some instances, the sealing membrane of the VCD may significantly reduce blood leakage from the vessel puncture, while complete hemostasis is achieved by a thrombus formed on or around the sealing membrane against the puncture. Thrombus forming capabilities may be enhanced by providing thrombus promoting materials on the sealing membrane and/or the anchoring tab or pull wire. The VCD may be left in the secured position within the vessel for essentially any period of time, which may be indefinitely in certain embodiments.

According to various embodiments, portions of the VCD are biodegradable, bioabsorbable, and/or bioerodable (collectively referred to herein as “biodegradable” unless expressly stated otherwise), such that after a period of time portions degrade, absorb, and/or erode. For example, at least the sealing membrane, and in some embodiments the support frame or portions thereof and/or an anchoring tab or pull wire, may degrade, dissolve, or become absorbed after a preselected period of time, minimizing the components remaining within the vessel over time. This may simplify subsequent access at or near the vessel puncture site and reduces potential long-term complications. The shape, configuration, and composition of the various components of the VCD, and the systems and methods for delivering the same, can be embodied in a number of manners, representative examples of which are described below.

The VCD described herein may be used to close punctures or penetrations in vessels in human or other animals (e.g., mammalian). Such an animal may be referred to herein as a patient. As used herein, the term “vessel” refers to arteries, veins, other vascular lumens for carrying blood or lymph, or other body lumens, such as, but not limited to, body lumens of the gastrointestinal system (e.g., the esophagus, the stomach, the small intestine, or the large intestine), the airway system (e.g., the trachea, the bronchus, or the bronchioles), the urinary system (e.g., the bladder, the ureters, or the urethra), or the cerebrospinal system (e.g., subarachnoid space or the ventricular system around and/or inside the brain and/or the spinal cord). The VCD can be dimensioned for effective use with a variety of vessel anatomies and sizes in adult and pediatric patients, as well as with punctures at a variety of vessel sites within the patient. It is envisioned that the VCD can be adapted for use in closing punctures in other body lumens in conjunction with various surgical procedures. For example, in one other embodiment, the VCD can be adapted for use to close lumen punctures during natural orifice transluminal endoscopic surgery or to close a lumbar puncture.

Vasculature Closure Devices and Methods of Delivery

Referring to the figures, FIG. 1 depicts an embodiment of the prior art VCD 100 implanted intraluminally within a patient's vessel 10 and positioned and secured therein to at least temporarily seal a target area at or near a vessel puncture site 15 (which is interchangeably referred to herein as the “access hole,” “access site,” “vessel puncture,” “puncture hole,” “puncture site,” or other similar variations thereof) existing through a wall of the vessel 10. According to this embodiment, the VCD 100 includes a sealing membrane 105 and an expandable support frame 110 providing shape and support to the sealing membrane 105 along at least a portion of the sealing membrane's 105 periphery. In other words, the sealing membrane 105 is at least partially supported by the support frame 110.

The support frame 110, and thus generally the VCD 100, is configured to expand from a collapsed configuration into an expanded configuration within the vessel 10. Upon expanding the support frame 110, the VCD 100 is configured to intraluminally position the sealing membrane 105 against the puncture site 15 to at least partially seal the puncture site 15. In some embodiments, as is shown in FIG. 1, the sealing membrane 105 and the support frame 110, and thus generally the VCD 100, may be formed in any shape configured for rolling and unrolling along a longitudinal axis generally aligned with and extending along the length of the lumen of the vessel 10 when implanted. The expansion of the VCD 100 thus may be in a radial direction i.e., perpendicular to the longitudinal axis, within the lumen of the vessel 10. For example, the VCD 100 may have a simple form that is similar in configuration to a sheet that can roll or unroll, or a tube that is slit entirely along its longitudinal axis. As described below, however, the VCD 100 may have any other shape that can be collapsed and then expanded within a vessel to promote securement of the VCD 100 therein.

According to the embodiment shown in FIG. 1, the VCD 100 also includes a cross-member support 115 extending across at least a portion of the sealing membrane 105. The cross-member support 115, due to its rigidity or at least partial rigidity, and/or tension provided by the peripheral support frame 110, provides structural and shape support to the sealing membrane 105. In some embodiments, the cross-member support 115 is more rigid than the sealing membrane 105. Upon expanding the support frame 110, the cross-member support 115 is configured to maintain the sealing membrane 105 against the puncture site 15, as is shown in FIG. 1. In other words, the cross-member support 115 may support the sealing membrane 105 to avoid sagging where the sealing membrane 105 bridges the puncture site 15, thus improving the seal created therebetween. In some embodiments, the cross-member support 115 extends between opposite sides of the support frame 110 and supports the sealing membrane 105 at or near a center of the sealing membrane 105 to avoid sagging at the puncture site 15. The cross-member support 115 also is configured to increase longitudinal rigidity of the VCD 100 during deployment into the vessel 10. In this manner, the cross-member support 115 may provide the longitudinal rigidity necessary for rolling the VCD 100 along the longitudinal axis and maintaining the VCD 100 in the collapsed configuration for deployment. In such embodiments, the VCD 100 may be configured for rolling and unrolling along a longitudinal axis defined by the cross-member support 115.

As is shown in the embodiment of FIG. 1, the VCD 100 further includes a tether, positioning tab, or anchoring tab 120 extending from the sealing membrane 105, the support frame 110, and/or the cross-member support 115. Specifically, the tether 120 is attached to at least one of the sealing membrane 105, the support frame 110, and/or the cross-member support 115, according to certain embodiments. Upon deployment of the VCD 100 within the vessel 10, the tether 120 extends out of and away from the puncture site 15. In this manner, the tether 120 may be pulled through and away from the puncture site 15 to position the sealing membrane 105 and the support frame 110 against an inner surface of the wall of the vessel 10 about the puncture site 15. Further, the tether 120 may facilitate intraluminal positioning or centering of the VCD 100 across the puncture site 15, as the VCD 100 may tend to migrate in a downstream direction (e.g., due to intravascular blood flow) toward a distal portion of the vessel 10 until the tether 120 abuts an edge of the vessel puncture 15. According to some embodiments, upon positioning the VCD 100 within the vessel 10, the free end portion of the tether 120 may be fixed to the patient, typically while the tether 120 is in tension. For example, the free end portion of the tether 120 may be affixed (e.g., sutured, glued, hooked, held by an elastic retaining means, etc.) to the patient's epidermis, dermis, sub-dermal layer, adipose layer, or muscle tissue at or near the vessel access site (e.g., at or near the initial incision created for access to the vessel).

It is appreciated that FIG. 1 is provided to depict one orientation of an embodiment of the VCD 100 within a vessel 10, and that any VCD according to the various embodiments described herein may be similarly positioned intraluminally to secure or otherwise retain a sealing membrane against a puncture site. These embodiments are described in more detail with reference to the figures.

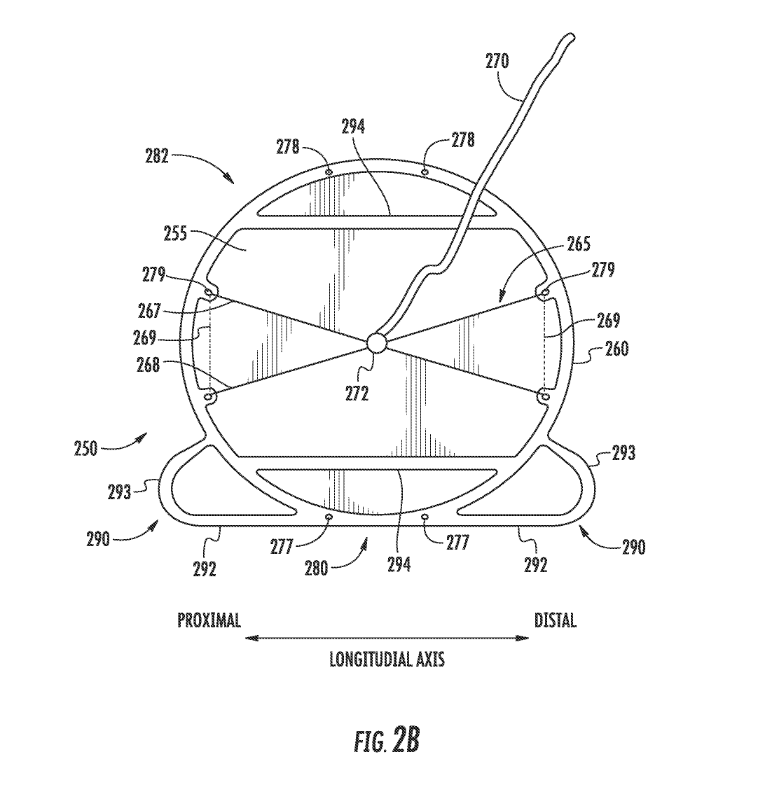

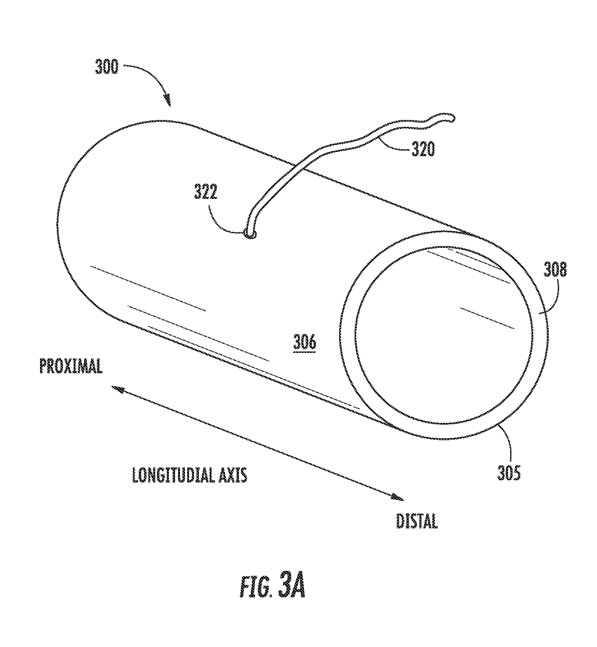

FIG. 2A illustrates one embodiment of a VCD 200, similar to the VCD 100 illustrated in and described with reference to FIG. 1, although certain differences in structure and function are described herein below. According to this embodiment, the VCD 200 includes a sealing membrane 205 and an expandable support frame 210 providing shape and support to the sealing membrane 205 along at least a portion of the sealing membrane's 205 periphery. In other words, the sealing membrane 205 is at least partially supported by the support frame 210. A particular difference between the VCD embodiments shown in FIG. 1 and FIG. 2A is that the embodiment of the VCD 200 shown in FIG. 2A includes a cross-member support 215 coupled to the sealing membrane 205 at an intermediate portion of the cross-member support 215. In this manner, the cross-member support 215 is configured to maintain the sealing membrane 205 against the puncture site 15 in the wall of the vessel 10. Notably, the VCD 200 is shown in FIG. 2A in a flat, fully unrolled position for illustration purposes only.

The support frame 210, and thus generally the VCD 200, is configured to expand from a collapsed configuration into an expanded configuration within the vessel 10. Upon expanding the support frame 210, the VCD 200 is configured to intraluminally position the sealing membrane 205 against the puncture site 15 to at least partially seal the puncture site 15. In some embodiments, the sealing membrane 205 and the support frame 210, and thus generally the VCD 200, are formed in any shape configured for rolling and unrolling along a longitudinal axis generally aligned with and extending along the length of the lumen of the vessel 10 when implanted. Specifically, the support frame 210 may be formed in a shape configured for rolling into the collapsed configuration and unrolling into the expanded configuration. The expansion of the VCD 200 thus may be in a radial direction i.e., perpendicular to the longitudinal axis, within the lumen of the vessel 10. For example, as is shown in FIG. 2A, the VCD 200 may have a simple form that is similar in configuration to a sheet that can roll or unroll. However, the VCD 200 may have any other shape that can be collapsed and then expanded within a vessel to promote securement of the VCD 200 therein.

According to the embodiment shown in FIG. 2A, the VCD 200 includes the cross-member support 215 extending across at least a portion of the sealing membrane 205. The cross-member support 215, due to its relative rigidity and/or tension provided by the peripheral support frame 210, provides structural and shape support to the sealing membrane 205. In some embodiments, the cross-member support 215 is more rigid than the sealing membrane 205. Upon expanding the support frame 210, the cross-member support 215 is configured to maintain the sealing membrane 205 against the puncture site 15. In other words, the cross-member support 215 supports the sealing membrane 205 to avoid sagging where the sealing membrane 205 bridges the puncture site 15, thus improving the seal created therebetween. In some embodiments, the cross-member support 215 extends between opposite sides of the support frame 210 and supports the sealing membrane 205 at or near a center of the sealing membrane 205 to avoid sagging at the puncture site 15. The cross-member support 215 also is configured to increase longitudinal rigidity of the VCD 200 during deployment into the vessel 10. In this manner, the cross-member support 215 may provide the longitudinal rigidity necessary for rolling the VCD 200 along the longitudinal axis and maintaining the VCD 200 in the collapsed configuration for deployment. In such embodiments, the VCD 200 may be configured for rolling and unrolling along a longitudinal axis defined by the cross-member support 215.

In certain embodiments, the cross-member support 215 is formed separately from and attached to the support frame 210. As is shown in FIG. 2A, the cross-member support 215 is attached to opposite sides of the support frame 210. In some embodiments, the cross-member support 215 extends over the sealing membrane 205 and is configured to be positioned between the sealing membrane 205 and the wall of the vessel 10. In other embodiments, the cross-member support 215 extends beneath the sealing membrane 205 and is configured to be positioned between the sealing membrane 205 and a flow of blood through the vessel 10. According to the embodiment shown in FIG. 2A, the cross-member support 215 is in the form of a flexible wire. In some embodiments, the flexible wire is formed of a surgical suture material. Examples of suitable materials of construction of the flexible wire include polymeric materials, such as PEEK (polyethylether ketone), fluorocarbon polymers, polyamides, polyimides, polyethylenes, polypropylenes, or similar polymers and copolymers. In some embodiments, the flexible wire is formed of a biodegradable material. Examples of suitable biodegradable materials of construction of the flexible wire include poly-L-lactide (PLLA), poly-D-lactide (PDLA), polyglycolide (PGA), poly(glycolic-co-lactic acid) (PLGA), polydioxanone (PDS), polycaprolactone (PCL), poly(glycolide-co-trimethylene carbonate) (PGA-TMC), polygluconate, and polylactic acid-polyethylene oxide.

As described above, the cross-member support 215 is coupled to the sealing membrane 205 at an intermediate portion of the cross-member support 215. In other words, the cross-member support 215 is coupled to the sealing membrane 205 at a portion between the ends of the cross-member support 215. In some embodiments, the intermediate portion of the cross-member support 215 is coupled to the sealing membrane 205 by a glue or solvent along one or more areas of the intermediate portion.

In other embodiments, as is shown in FIG. 2A, the intermediate portion of the cross-member support 215 is coupled to the sealing membrane 205 by a coupler 218 attached to the sealing membrane 205 and extending over the intermediate portion of the cross-member support 215. The coupler 218 may be attached to the sealing membrane 205 by glue, solvent adhesion, thermal welding, ultrasonic welding, laser welding, or any other coupling technique known to those skilled in the art. The coupler 218 may be positioned at any point along the longitudinal axis of the cross-member support 215. In some embodiments, the coupler 218 is positioned at about a center of the longitudinal axis of the cross-member support 215 and/or at about a center of the longitudinal axis of the VCD 200. In some embodiments, the intermediate portion of the cross-member support 215 is fixed between the coupler 218 and the sealing membrane 205. In other embodiments, the intermediate portion of the cross-member support 215 is movable between the coupler 218 and the sealing membrane 205. In such embodiments, the coupler 218 may be attached to the sealing membrane 205 only at the ends of the coupler 218, while the center portion of the coupler 218 remains free so that the cross-member support 215 may have some space to move.

The coupler 218 may be in the form of a wire having a cross-section of about 0.025 mm to about 1 mm, and preferably of about 0.05 mm to about 0.2 mm. Alternatively, as is shown in FIG. 2A, the coupler 218 is in the form of a patch having a width of about 0.2 mm to about 15 mm and a length of about 1 mm to about 15 mm. In another embodiment, the patch has a width of about 1 mm to about 5 mm and a length of about 2 mm to about 8 mm. The thickness of the patch may range from about 1 μm to about 250 μm, preferably from about 10 μm to about 150 μm, and more preferably from about 20 μm to about 60 μm. In some embodiments, the coupler 218 is formed of a biodegradable material, such as PLLA, PDLA, PGA, PLGA, PDS, PCL, PGA-TMC, polygluconate, or polylactic acid-polyethylene oxide. In one embodiment, the coupler 218 is formed of the same material as the sealing membrane 205.

As is shown in the embodiment of FIG. 2A, the VCD 200 further includes a tether, positioning tab, or anchoring tab 220 extending from the sealing membrane 205, the support frame 210, and/or the cross-member support 215. Specifically, the tether 220 is attached to at least one of the sealing membrane 205, the support frame 210, and/or the cross-member support 215, according to certain embodiments. In some embodiments, as is shown in FIG. 2A, the tether 220 is attached to the cross-member support 215 at a securing point 222. The securing point 222 may be at about the center of the longitudinal axis of the cross-member support 215 and/or at about the center of the longitudinal axis of the VCD 200. Alternatively, the securing point 222 may be proximal or distal to the center of the longitudinal axis of the cross-member support 215 and/or the center of the longitudinal axis of the VCD 200. In a preferred embodiment, the securing point 222 is between 5 mm distal and 5 mm proximal to the center of the longitudinal axis of the VCD 200. In some embodiments, the coupler 218 is positioned in proximity to the securing point 222. The coupler 218 may be positioned in a proximal or distal location relative to the securing point 222. In some embodiments, the VCD 200 includes two or more couplers 218, some positioned proximal to and some positioned distal to the securing point 222.

Upon deployment of the VCD 200 within the vessel 10, the tether 220 extends out of and away from the puncture site 15. In this manner, the tether 220 may be pulled through and away from the puncture site 15 to position the sealing membrane 205 and the support frame 210 against an inner surface of the wall of the vessel 10 about the puncture site 15. Further, the tether 220 may facilitate intraluminal positioning or centering of the VCD 200 across the puncture site 15, as the VCD 200 may tend to migrate in a downstream direction toward a distal portion of the vessel 10 until the tether 220 abuts an edge of the vessel puncture 15. According to some embodiments, upon positioning the VCD 200 within the vessel 10, the free end portion of the tether 220 may be affixed to the patient in a like manner as tether 120 described above with reference to FIG. 1.

As is shown in the embodiment of FIG. 2A, the support frame 210 is formed as a peripheral support frame defining an oval shape, although the peripheral support frame may define a circular shape in other embodiments. The sealing membrane 205 may define an outer edge about its periphery, and at least a portion of the support frame 210 may be positioned along the outer edge of the sealing membrane 205. In some embodiments, the outer edge of the sealing membrane 205 extends beyond the outer edges of the support frame 210. The sealing membrane 205 may be attached to the support frame 210 using glue, solvent adhesion, laser welding, ultrasonic welding, thermal welding, or any other means of attachment. In some embodiments, the sealing membrane 205 includes a plurality of tabs extending about the outer edge, and the sealing membrane 205 is attached to the support frame 210 by the plurality of tabs. Specifically, each of the tabs may wrap around a portion of the support frame 210 and be bonded to the sealing membrane 205 or wrapped around the support frame 210 and bonded to itself. In other embodiments, as is shown in FIG. 2A, the support frame 210 defines a plurality of holes 227, 228 for attaching the sealing membrane 205 to the support frame 210. Specifically, the sealing membrane 205 may be attached to the support frame 210 by a plurality of anchors extending through the plurality of holes 227, 228. The anchors may be formed of a glue or adhesive used to fill the holes 227, 228 until reaching the membrane 205, such that the cured glue or adhesive forms a stud-like shape extending through the holes 227, 228 and holding the sealing membrane 205 to the support frame 210. Alternatively, the anchors may be formed of the same material as the sealing membrane 205, for example by casting, such that the material forms a stud-like shape extending through the holes 227, 228 and holding the sealing membrane 205 to the support frame 210. Further, the anchors may be formed as a wire, such as a surgical suture material, or a rivet type fastener extending through the plurality of holes 227, 228. In still other embodiments, the support frame 210 is integrated with the sealing membrane 205 during manufacturing. The integrated configuration may be formed, for example, by depositing or casting an initial layer of the sealing membrane 205, placing the support frame 210 onto the initial layer of the sealing membrane 210, and then depositing or casting a second layer of the sealing membrane 205 onto the initial layer and the support frame 210, such that the support frame 210 is embedded within the sealing membrane 205. In some embodiments, as is shown in FIG. 2A, the support frame 210 defines a plurality of holes 229 for attaching the cross-member support 215 to the support frame 210.

As discussed above, the support frame 210 is configured to expand from a collapsed configuration into an expanded configuration within the vessel 10. Specifically, the support frame 210 may be configured to expand from the collapsed configuration having a first radius of curvature into the expanded configuration having a second radius of curvature greater than the first radius of curvature. In some embodiments, the support frame 210 is configured to expand into the expanded configuration having a radius of curvature greater than a radius of curvature of the vessel 10. In some embodiments, the support frame 210 is formed of a self-expandable or pre-shaped material having a pre-shaped expanded configuration, such that the support frame 210 tends to assume the pre-shaped expanded configuration absent the application of external forces to the support frame 210. In this manner, the support frame 210 may be configured to self-expand from the collapsed configuration into the pre-shaped expanded configuration within the vessel 10 upon deployment or release of the VCD 200 from a containment mechanism (and consequent release of a compressive load holding the VCD 200 in the collapsed configuration). The pre-shaped material may include a shape memory metal and/or a shape memory polymer, and the pre-shaped expanded configuration of the support frame 210 may be defined by the stable shape of the shape memory metal and/or shape memory polymer. Preferably, the support frame 210 is formed of a nickel-titanium alloy. Other elastic or super-elastic materials may be used to form the support frame 210.

As discussed above, the support frame 210 is configured for rolling into the collapsed configuration and unrolling into the expanded configuration. In some embodiments, as is shown in FIG. 2A, the support frame 210 includes a first wing 230 and a second wing 232 positioned opposite the first wing 230. In this manner, the second wing 232 may be rolled over the first wing 230 when the support frame 210 is in the collapsed configuration. The support frame 210 also may include at least one, and preferably two, tabs 240 extending from the first wing 230. The tabs 240 may provide multiple utilities. First, the tabs 240 may be configured to increase a longitudinal stiffness of the VCD 200 when the support frame 210 is in the collapsed configuration during delivery of the VCD 200. Specifically, in some embodiments, each of the tabs 240 includes a straight segment 242 extending along the longitudinal axis of the VCD 200, which serves as a longitudinal stiffener. Second, the tabs 240 may be configured to prevent the first wing 230 from applying pressure on the sealing membrane 205 when the support frame 210 is in the collapsed configuration. Specifically, in some embodiments, each of the tabs 240 includes a curved segment 243 configured to contact a portion of the support frame 210 that is rolled over the tabs 240 when the support frame 210 is in the collapsed configuration, such that the first wing 230 does not contact the sealing membrane 205. The curved segment 243 may be configured to contact the support frame 210 at or near the centerline of the support frame 210 (i.e., between the first wing 230 and the second wing 232). Third, the tabs 240 may be configured to apply a force to the portion of the support frame 210 that is rolled over the tabs 240 for unrolling the support frame 210 into the expanded configuration. Specifically, in some embodiments, the curved segments 243 of the tabs 240 are configured to apply an expansion force to the support frame 210 at or near the centerline of the support frame 210 such that the support frame 210 self-expands from the collapsed configuration into the pre-shaped expanded configuration. In the absence of the tabs 240, and specifically the curved segments 243 of the tabs 240, the expansion force would be applied by the first wing 230 to the sealing membrane 205, which may result in damage or unwanted deformation to the sealing membrane 205 or penetration of the first wing 230 into the sealing membrane 205 and which may significantly increase the force needed to expand the support frame 210, possibly to a level such that the support frame 210 may not be able to return to its expanded configuration upon release of the containment mechanism. In some embodiments, as is shown in FIG. 2A, the support frame 210 further includes one or more longitudinal supports 244 extending longitudinally between opposite sides of the support frame 210. In this manner, the longitudinal supports 244 are configured to increase a longitudinal stiffness of the VCD 200, particularly when the support frame 210 is in the collapsed configuration during delivery of the VCD 200.

FIG. 2B illustrates one embodiment of a VCD 250, similar to the VCD 200 illustrated in and described with reference to FIG. 2A, although certain differences in structure and function are described herein below. According to this embodiment, the VCD 250 includes a sealing membrane 255 and an expandable support frame 260 providing shape and support to the sealing membrane 255 along at least a portion of the sealing membrane's 255 periphery. In other words, the sealing membrane 255 is at least partially supported by the support frame 260. A particular difference between the VCD embodiments shown in FIG. 2A and FIG. 2B is that the embodiment of the VCD 250 shown in FIG. 2B includes a cross-member support 265 in the form of a flexible wire including wire segments 267, 268 that define an X-shape of the cross-member support 265. In this manner, the wire segments 267, 268 are configured to distribute forces applied to the cross-member support 265. Notably, the VCD 250 is shown in FIG. 2B in a flat, fully unrolled position for illustration purposes only.

The support frame 260, and thus generally the VCD 250, is configured to expand from a collapsed configuration into an expanded configuration within the vessel 10. Upon expanding the support frame 260, the VCD 250 is configured to intraluminally position the sealing membrane 255 against the puncture site 15 to at least partially seal the puncture site 15. In some embodiments, the sealing membrane 255 and the support frame 260, and thus generally the VCD 250, are formed in any shape configured for rolling and unrolling along a longitudinal axis generally aligned with and extending along the length of the lumen of the vessel 10 when implanted. Specifically, the support frame 260 may be formed in a shape configured for rolling into the collapsed configuration and unrolling into the expanded configuration. The expansion of the VCD 250 thus may be in a radial direction i.e., perpendicular to the longitudinal axis, within the lumen of the vessel 10. For example, as is shown in FIG. 2B, the VCD 250 may have a simple form that is similar in configuration to a sheet that can roll or unroll. However, the VCD 250 may have any other shape that can be collapsed and then expanded within a vessel to promote securement of the VCD 250 therein.

According to the embodiment shown in FIG. 2B, the VCD 250 includes the cross-member support 265 extending across at least a portion of the sealing membrane 255. The cross-member support 265, due to its relative rigidity and/or tension provided by the peripheral support frame 260, provides structural and shape support to the sealing membrane 255. In some embodiments, the cross-member support 265 is more rigid than the sealing membrane 255. Upon expanding the support frame 260, the cross-member support 265 is configured to maintain the sealing membrane 255 against the puncture site 15. In other words, the cross-member support 265 supports the sealing membrane 255 to avoid sagging where the sealing membrane 255 bridges the puncture site 15, thus improving the seal created therebetween. In some embodiments, the cross-member support 265 extends between opposite sides of the support frame 260 and supports the sealing membrane 255 at or near a center of the sealing membrane 255 to avoid sagging at the puncture site 15. The cross-member support 265 also is configured to increase longitudinal rigidity of the VCD 250 during deployment into the vessel 10. In this manner, the cross-member support 265 may provide the longitudinal rigidity necessary for rolling the VCD 250 along the longitudinal axis and maintaining the VCD 250 in the collapsed configuration for deployment. In such embodiments, the VCD 250 may be configured for rolling and unrolling along a longitudinal axis defined by the cross-member support 265.

In certain embodiments, the cross-member support 265 is formed separately from and attached to the support frame 260. As is shown in FIG. 2B, the cross-member support 265 is attached to opposite sides of the support frame 260. In some embodiments, the cross-member support 265 extends over the sealing membrane 255 and is configured to be positioned between the sealing membrane 255 and the wall of the vessel 10. In other embodiments, the cross-member support 265 extends beneath the sealing membrane 255 and is configured to be positioned between the sealing membrane 255 and a flow of blood through the vessel 10. According to the embodiment shown in FIG. 2B, the cross-member support 265 is in the form of a flexible wire. In some embodiments, the flexible wire is formed of a surgical suture material. Examples of suitable materials of construction of the flexible wire include polymeric materials, such as PEEK, fluorocarbon polymers, polyamides, polyimides, polyethylenes, polypropylenes, or similar polymers and copolymers. In some embodiments, the flexible wire is formed of a biodegradable material. Examples of suitable biodegradable materials of construction of the flexible wire include PLLA, PDLA, PGA, PLGA, PDS, PCL, PGA-TMC, polygluconate, and polylactic acid-polyethylene oxide.

According to the embodiment shown in FIG. 2B, the cross-member support 215 is in the form of a flexible wire including a first wire segment 267 extending between opposite sides of the support frame 260 and a second wire segment 268 extending between opposite sides of the support frame 260. As is shown, the first wire segment 267 and the second wire segment 268 define an X-shape of the cross-member support 265, such that the first wire segment 267 and the second wire segment 268 are configured to distribute forces applied to the cross-member support 265. In some embodiments, the first wire segment 267 and the second wire segment 268 are formed of a single flexible wire and are connected to one another by one or more additional wire segments 269, as is shown by dashed lines. In other embodiments, the first wire segment 267 and the second wire segment 268 are formed of separate wires. In some embodiments, the cross-member support 265 extends over the sealing membrane 255 and is configured to be positioned between the sealing membrane 255 and the wall of the vessel 10. In other embodiments, the cross-member support 265 extends beneath the sealing membrane 255 and is configured to be positioned between the sealing membrane 255 and a flow of blood through the vessel 10.

In some embodiments, the cross-member support 265 is coupled to the sealing membrane 255 at an intermediate portion of the cross-member support 265. In other words, the cross-member support 265 is coupled to the sealing membrane 255 at a portion between the ends of the cross-member support 265. In some embodiments, the intermediate portion of the cross-member support 265 is coupled to the sealing membrane 255 by a glue or solvent along one or more areas of the intermediate portion. In other embodiments, the intermediate portion of the cross-member support 265 is coupled to the sealing membrane 255 by a coupler (not shown) in a like manner as coupler 218 described above with reference to FIG. 2A.

As is shown in the embodiment of FIG. 2B, the VCD 250 further includes a tether, positioning tab, or anchoring tab 270 attached to the cross-member support 265. Specifically, the tether 270 may be attached to the cross-member support 265 at a securing point 272 at a center of the X-shape of the cross-member support 265. In this manner, the first wire segment 267 and the second wire segment 268 may be configured to distribute pulling forces applied to the cross-member support 265 via the tether 270 to reduce bending of the support frame 260. Accordingly, greater pulling forces may be applied during positioning of the VCD 250 within the vessel before significant bending or deformation of the support frame 260 occurs. The securing point 272 may be at about the center of the longitudinal axis of the VCD 250. Alternatively, the securing point 272 may be at about 1 mm to about 6 mm proximal to or at about 1 mm to about 6 mm distal to the center of the longitudinal axis of the VCD 250.

Upon deployment of the VCD 250 within the vessel 10, the tether 270 extends out of and away from the puncture site 15. In this manner, the tether 270 may be pulled through and away from the puncture site 15 to position the sealing membrane 255 and the support frame 260 against an inner surface of the wall of the vessel 10 about the puncture site 15. Further, the tether 270 may facilitate intraluminal positioning or centering of the VCD 250 across the puncture site 15, as the VCD 250 may tend to migrate in a downstream direction toward a distal portion of the vessel 10 until the tether 270 abuts an edge of the vessel puncture 15. According to some embodiments, upon positioning the VCD 250 within the vessel 10, the free end portion of the tether 270 may be affixed to the patient in a like manner as tether 120 described above with reference to FIG. 1.

As is shown in the embodiment of FIG. 2B, the support frame 260 is formed as a peripheral support frame defining an oval shape, although the peripheral support frame may define a circular shape in other embodiments. The sealing membrane 255 may define an outer edge about its periphery, and at least a portion of the support frame 260 may be positioned along the outer edge of the sealing membrane 255. In some embodiments, the outer edge of the sealing membrane 255 extends beyond the outer edges of the support frame 260. The sealing membrane 255 may be attached to the support frame 260 using glue, solvent adhesion, laser welding, ultrasonic welding, thermal welding, or any other means of attachment. In some embodiments, the sealing membrane 255 includes a plurality of tabs extending about the outer edge, and the sealing membrane 255 is attached to the support frame 260 by the plurality of tabs. Specifically, each of the tabs may wrap around a portion of the support frame 260 and be bonded to the sealing membrane 255 or wrapped around the support frame 260 and bonded to itself. In other embodiments, as is shown in FIG. 2B, the support frame 270 defines a plurality of holes 277, 278 for attaching the sealing membrane 255 to the support frame 260. Specifically, the sealing membrane 255 may be attached to the support frame 260 by a plurality of anchors extending through the plurality of holes 277, 278. The anchors may be formed of a glue or adhesive used to fill the holes 277, 278 until reaching the membrane 255, such that the cured glue or adhesive forms a stud-like shape extending through the holes 277, 278 and holding the sealing membrane 255 to the support frame 260. Alternatively, the anchors may be formed of the same material as the sealing membrane 255, for example by casting, such that the material forms a stud-like shape extending through the holes 277, 278 and holding the sealing membrane 255 to the support frame 260. Further, the anchors may be formed as a wire, such as a surgical suture material, or a rivet type fastener extending through the plurality of holes 277, 278. In still other embodiments, the support frame 260 is integrated with the sealing membrane 255 during manufacturing. The integrated configuration may be formed, for example, by depositing or casting an initial layer of the sealing membrane 255, placing the support frame 260 onto the initial layer of the sealing membrane 260, and then depositing or casting a second layer of the sealing membrane 255 onto the initial layer and the support frame 260, such that the support frame 260 is embedded within the sealing membrane 255. In some embodiments, as is shown in FIG. 2B, the support frame 260 defines a plurality of holes 279 for attaching the cross-member support 265 to the support frame 260.

As discussed above, the support frame 260 is configured to expand from a collapsed configuration into an expanded configuration within the vessel 10. Specifically, the support frame 260 may be configured to expand from the collapsed configuration having a first radius of curvature into the expanded configuration having a second radius of curvature greater than the first radius of curvature. In some embodiments, the support frame 260 is configured to expand into the expanded configuration having a radius of curvature greater than a radius of curvature of the vessel 10. In some embodiments, the support frame 260 is formed of a self-expandable or pre-shaped material having a pre-shaped expanded configuration, such that the support frame 260 tends to assume the pre-shaped expanded configuration absent the application of external forces to the support frame 260. In this manner, the support frame 260 may be configured to self-expand from the collapsed configuration into the pre-shaped expanded configuration within the vessel 10 upon deployment or release of the VCD 250 from a containment mechanism (and consequent release of a compressive load holding the VCD 250 in the collapsed configuration). The pre-shaped material may include a shape memory metal and/or a shape memory polymer, and the pre-shaped expanded configuration of the support frame 250 may be defined by the stable shape of the shape memory metal and/or shape memory polymer. Preferably, the support frame 260 is formed of a nickel-titanium alloy. Other elastic or super-elastic materials may be used to form the support frame 260.

As discussed above, the support frame 260 is configured for rolling into the collapsed configuration and unrolling into the expanded configuration. In some embodiments, as is shown in FIG. 2B, the support frame 260 includes a first wing 280 and a second wing 282 positioned opposite the first wing 280. In this manner, the second wing 282 may be rolled over the first wing 280 when the support frame 260 is in the collapsed configuration. The support frame 260 also may include at least one, and preferably two, tabs 290 extending from the first wing 280. The tabs 290 may provide multiple utilities. First, the tabs 290 may be configured to increase a longitudinal stiffness of the VCD 250 when the support frame 260 is in the collapsed configuration during delivery of the VCD 250. Specifically, in some embodiments, each of the tabs 290 includes a straight segment 292 extending along the longitudinal axis of the VCD 250, which serves as a longitudinal stiffener. Second, the tabs 290 may be configured to prevent the first wing 280 from applying pressure on the sealing membrane 255 when the support frame 260 is in the collapsed configuration. Specifically, in some embodiments, each of the tabs 290 includes a curved segment 293 configured to contact a portion of the support frame 260 that is rolled over the tabs 290 when the support frame 260 is in the collapsed configuration, such that the first wing 280 does not contact the sealing membrane 255. The curved segment 293 may be configured to contact the support frame 260 at or near the centerline of the support frame 260 (i.e., between the first wing 280 and the second wing 282). Third, the tabs 290 may be configured to apply a force to the portion of the support frame 260 that is rolled over the tabs 290 for unrolling the support frame 260 into the expanded configuration. Specifically, in some embodiments, the curved segments 293 of the tabs 290 are configured to apply an expansion force to the support frame 260 at or near the centerline of the support frame 260 such that the support frame 260 self-expands from the collapsed configuration into the pre-shaped expanded configuration. In the absence of the tabs 290, and specifically the curved segments 293 of the tabs 290, the expansion force would be applied by the first wing 280 to the sealing membrane 255, which may result in damage or unwanted deformation to the sealing membrane 255 or penetration of the first wing 280 into the sealing membrane 255 and which may significantly increase the force needed to expand the support frame 260, possibly to a level such that the support frame 260 may not be able to return to its expanded configuration upon release of the containment mechanism. In some embodiments, as is shown in FIG. 2B, the support frame 260 further includes one or more longitudinal supports 294 extending longitudinally between opposite sides of the support frame 260. In this manner, the longitudinal supports 294 are configured to increase a longitudinal stiffness of the VCD 250, particularly when the support frame 260 is in the collapsed configuration during delivery of the VCD 250.

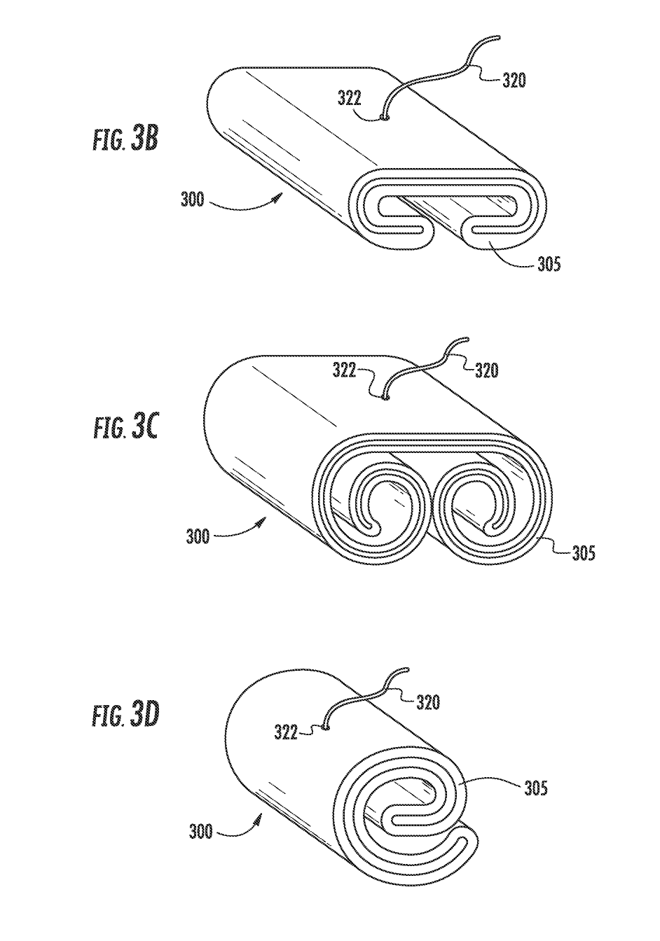

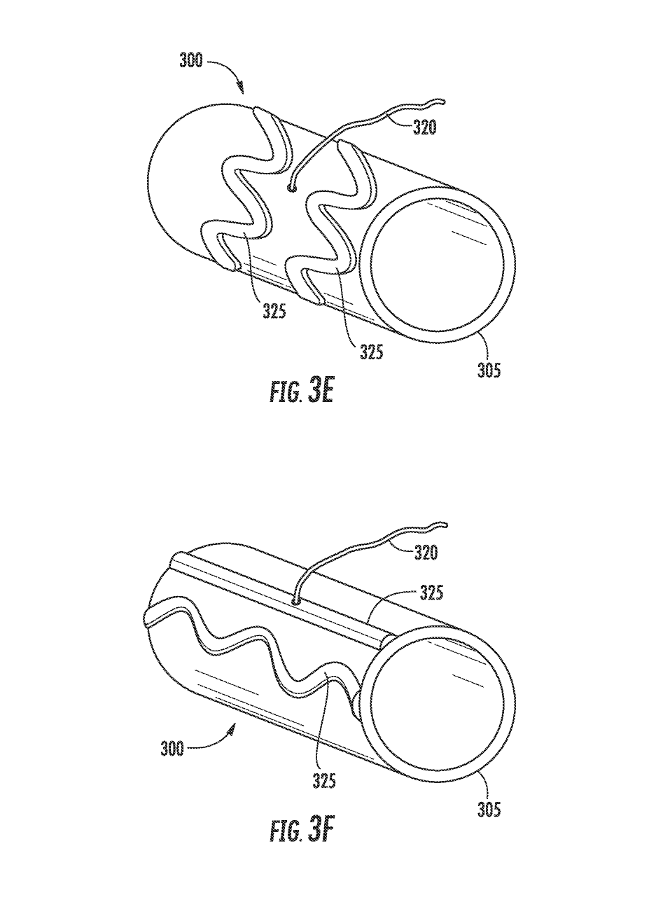

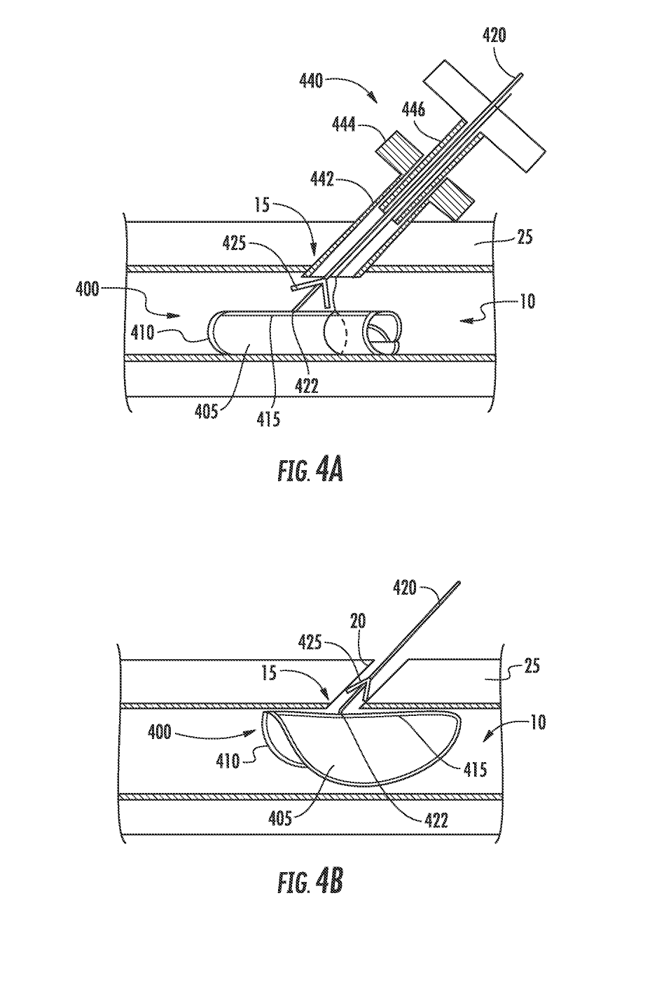

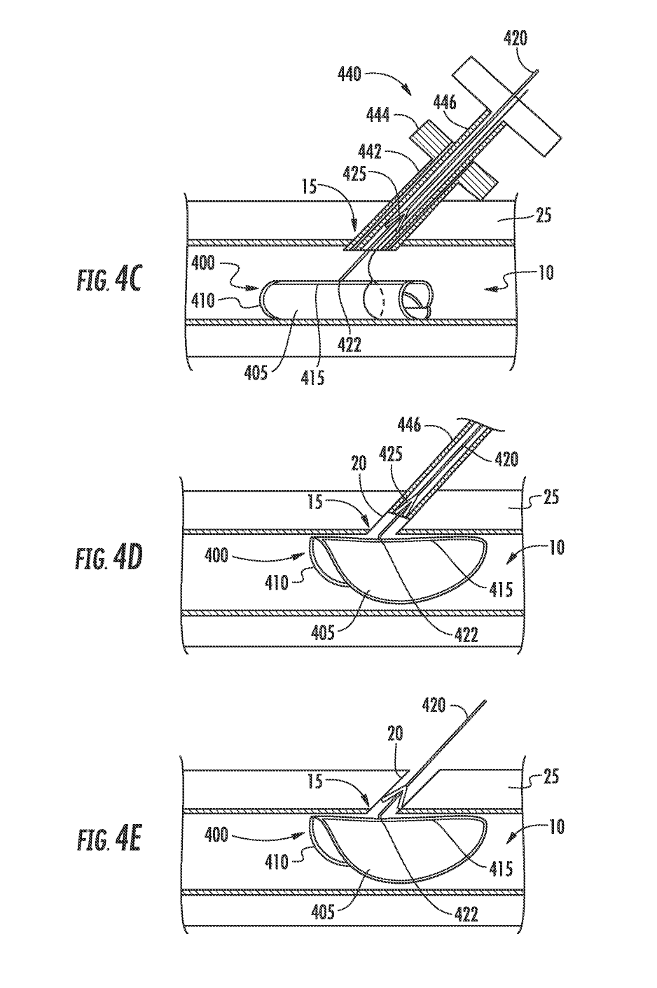

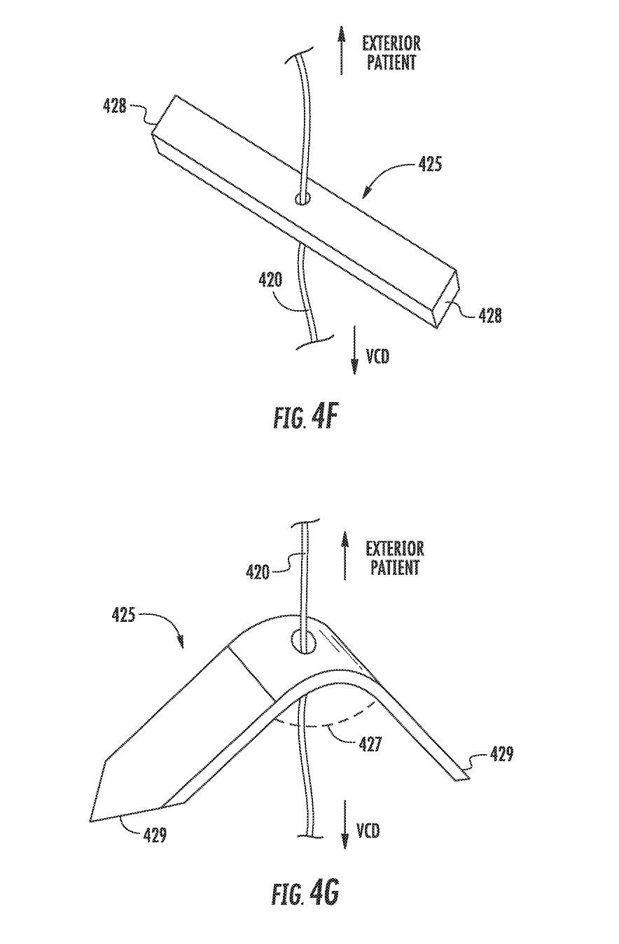

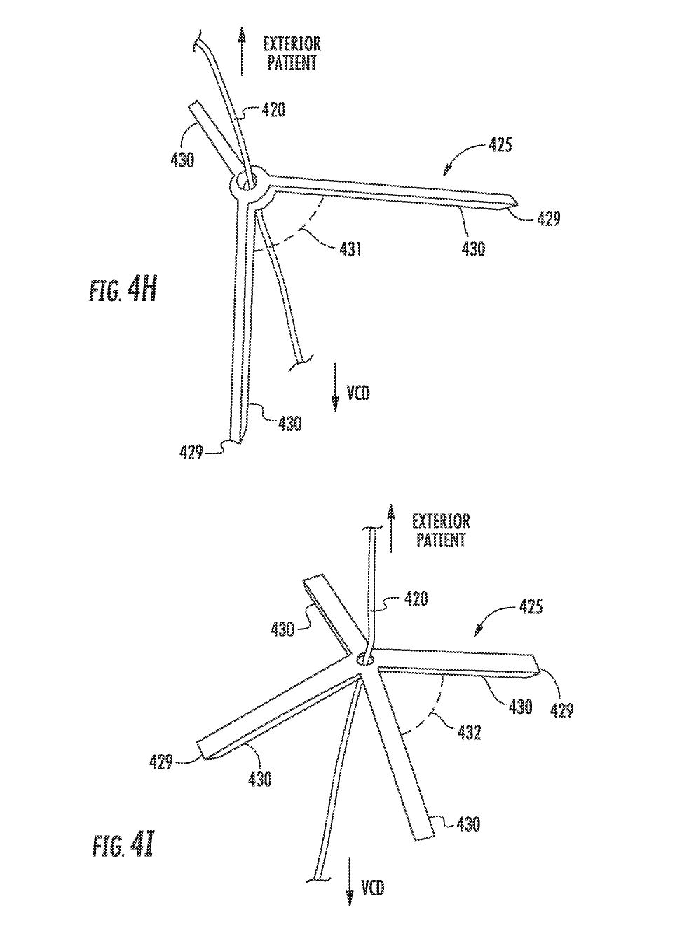

FIG. 3A illustrates one embodiment of a VCD 300, similar to the VCD 100 illustrated in and described with reference to FIG. 1, although certain differences in structure and function are described herein below. A particular difference between the VCD embodiments shown in FIG. 1 and FIG. 3A is that the embodiment of the VCD 300 shown in FIG. 3A includes an expandable tube 305 deployable within the vessel 10. The tube 305 is configured to expand from a collapsed configuration into an expanded configuration within the vessel 10 to intraluminally position an outer surface 306 of the tube against the puncture site 15 to at least partially seal the puncture site 15. As is shown in FIG. 3A, the tube 305 includes a solid sidewall 308 defining the outer surface 306 for sealing the puncture site 15. In other words, the sidewall 308 of the tube 305 does not include any openings extending therethrough, such that the outer surface 306 is suitable for sealing the puncture site 15.

As is shown in the embodiment of FIG. 3A, the VCD 300 further includes a tether, positioning tab, or anchoring tab 320 extending from the tube 305. Specifically, the tether 320 may be attached to the outer surface 306 of the tube 305 at a securing point 322. The securing point 322 may be at about the center of the longitudinal axis of the tube 305 and/or the center of the longitudinal axis of the VCD 300. Alternatively, the securing point 322 may be proximal or distal to the center of the longitudinal axis of the tube 305 and/or the center of the longitudinal axis of the VCD 300. In a preferred embodiment, the securing point 322 is between 5 mm distal and 5 mm proximal to the center of the longitudinal axis of the VCD 300. Upon deployment of the VCD 300 within the vessel 10, the tether 320 extends out of and away from the puncture site 15. In this manner, the tether 320 may be pulled through and away from the puncture site 15 to position the outer surface of the tube 305 against an inner surface of the wall of the vessel 10 about the puncture site 15. Further, the tether 320 may facilitate intraluminal positioning or centering of the VCD 300 across the puncture site 15, as the VCD 300 may tend to migrate in a downstream direction toward a distal portion of the vessel 10 until the tether 320 abuts an edge of the vessel puncture 15. According to some embodiments, upon positioning the VCD 300 within the vessel 10, the free end portion of the tether 320 may be affixed to the patient in a like manner as the tether 120 described above with reference to FIG. 1.

As discussed above, the tube 305 is configured to expand from a collapsed configuration into an expanded configuration within the vessel 10. Specifically, the tube 305 may be configured to expand from the collapsed configuration having a first radial profile into the expanded configuration having a second radial profile greater than the first radial profile. In some embodiments, the tube 305 is configured to expand into the expanded configuration having an outer diameter substantially equal to the inner diameter of the vessel 10. In other embodiments, the tube 305 is configured to expand into the expanded configuration having an outer diameter greater than the inner diameter of the vessel 10. The VCD 300 may be designed and configured for the closure of 12 Fr to 27 Fr sheath punctures having a diameter of about 4.5 mm to about 10 mm, and thus the collapsed configuration of the tube 305 may have a radial profile smaller than the diameter of the sheath puncture. Further, the collapsed configuration of the tube 305 may have a radial profile smaller than an inner diameter of a delivery sheath.

In some embodiments, the tube 305 is formed of a self-expandable or pre-shaped material having a pre-shaped expanded configuration, such that the tube 305 tends to assume the pre-shaped expanded configuration absent the application of external forces to the tube 305. In this manner, the tube 305 may be configured to self-expand from the collapsed configuration into the pre-shaped expanded configuration within the vessel 10 upon deployment or release of the VCD 300 from a containment mechanism. In some embodiments, the tube 305 is configured to self-expand over a time scale of seconds to minutes. Accordingly, in such embodiments, the VCD 300 may be delivered without a containment mechanism, as the tube 305 may retain its collapsed configuration during the short time of delivery, and positioning of the VCD 300 within the lumen of the vessel 10 may be achieved before the tube 305 self-expands into the pre-shaped expanded configuration. In such embodiments, the tether 320 may be kept under tension to assure coupling of the VCD 300 across the puncture site 15, preventing migration of the tube 305 and minimizing bleeding until the tube 305 expands into the pre-shaped expanded configuration for sealing the puncture site 15. The tube 305 is preferably formed of a polymeric material, and more preferably formed of a biodegradable polymer. Examples of suitable materials of construction of the tube 305 include but are not limited to polyester (e.g., PLLA, PDLA, PGA, or PLGA), PDS, PCL, PGA-TMC, polygluconate, polylactic acid-polyethylene oxide copolymers, poly(hydroxybutyrate), polyanhydride, polyphosphoester, poly(amino acids), poly(alpha-hydroxy acid), or any other similar copolymers. In one embodiment, the tube 305 is formed of biodegradable poly(ε-caprolact+one-co-DL-lactide) copolymer and has a wall thickness of about 1 mm. Yu, et al.; J. Material Science: Material in Medicine, 23(2):581-89 (February 2012) discloses the use of biodegradable poly(ε-caprolactone-co-DL-lactide) copolymer as a building material for a esophageal stent.

FIGS. 3B-3D illustrate three possible collapsed configurations of the tube 305 for percutaneous delivery of the VCD 300 into the vessel 10. In some embodiments, the tube 305 is configured to be flattened into the collapsed configuration. As is shown in FIG. 3B, the tube 305 first may be flattened and then opposite sides of the flattened tube 305 may be folded inward toward the center of the tube 305. Alternatively, as is shown in FIG. 3C, the tube 305 first may be flattened and then opposite sides of the flattened tube 305 may be rolled inward toward the center of the tube 305. Further, as is shown in FIG. 3D, the tube 305 first may be flattened, then a first side of the flattened tube 305 may be folded inward toward the center of the tube 305, and then a second side of the flattened tube 305 may be rolled over the first side. This method of collapsing the tube 305 beneficially provides a more rounded and compact collapsed configuration of the tube 305, resulting in a smaller radial profile. As is shown in FIGS. 3B-3D, when the tube 305 is in the collapsed configuration, the securing point 322 of the tether 320 preferably is positioned on an upper side of the collapsed tube 305, such that no layer of the tube 305 is rolled or folded over the securing point 322. This positioning advantageously enables the physician during delivery of the VCD 300 to pull the tether 320 and position the upper side of the tube 305 against the puncture site 15 without interfering with expansion (e.g., unrolling and/or unfolding) of the tube 305.

In some embodiments, the sidewall 308 of the tube 305 has a uniform wall thickness along the length and circumference of the tube 305. In other embodiments, the sidewall 308 of the tube 305 has a wall thickness that varies along the length and/or circumference of the tube 305, resulting in areas of substantially greater wall thickness than in other thinner areas. The areas of greater wall thickness may provide enhanced rigidity or support to the tube 305 structure radially, longitudinally, or both. In some embodiments, as is shown in FIGS. 3E and 3F, the VCD 300 includes at least one supporting element 325 extending along the tube and configured to increase rigidity of the tube 305. The supporting element 325, which may be in the form of one or more ribs, may be positioned on the outer surface 306 and/or the inner surface of the tube 305. Alternatively, the supporting element 325 may be positioned between the outer surface 306 and the inner surface of the tube 305. As is shown in FIG. 3E, the supporting element 325 is a radial supporting element extending along the circumference of the tube 305 and configured to increase the radial rigidity of the tube 305. Additionally, when formed as a radial supporting element, the supporting element 325 may facilitate expansion of the VCD 300 from the collapsed configuration into the expanded configuration. Alternatively, as is shown in FIG. 3F, the supporting element 325 is a longitudinal supporting element extending along the length of the tube 305 and configured to increase the longitudinal rigidity of the tube 305. In some embodiments, the VCD 300 includes a plurality of supporting elements 325, which may include a plurality of radial supporting elements, a plurality of longitudinal supporting elements, or at least one radial supporting element and at least one longitudinal supporting element. The supporting element 325 may define a straight shape along the circumference or the length of the tube 305. Alternatively, the supporting element 325 may define a zig-zag or sinusoidal shape along the circumference or the length of the tube 305. Other shapes may be used for increasing the rigidity of the tube 305, as known to those skilled in the art of stent design. In some embodiments, the supporting element 325 is integrally formed with the tube 305. In other embodiments, the supporting element 325 is formed separately from and attached to the tube 305.

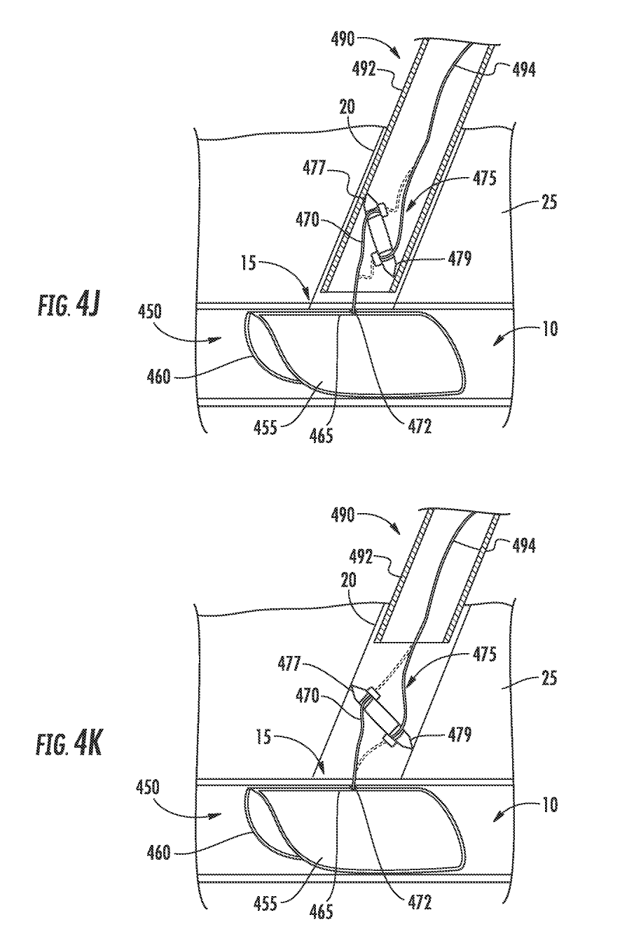

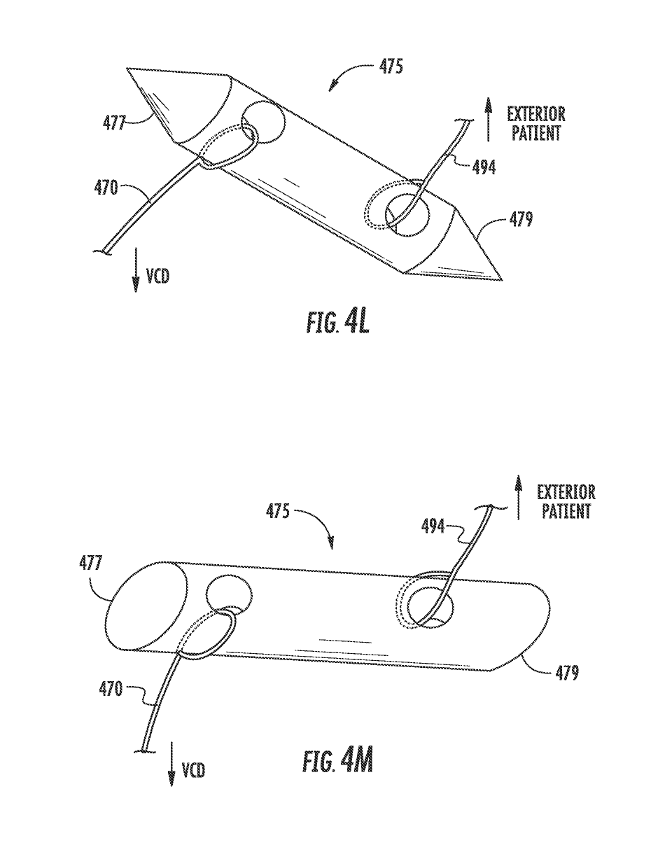

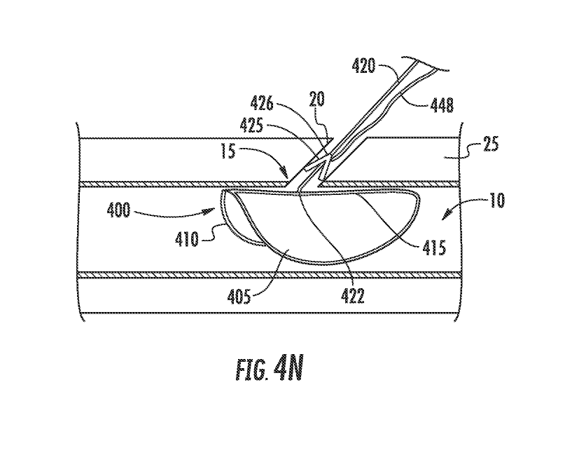

FIGS. 4A and 4B illustrate one embodiment of a VCD 400, similar to the VCD 100 illustrated in and described with reference to FIG. 1, although certain differences in structure and function are described herein below. According to this embodiment, the VCD 400 includes a sealing membrane 405 and an expandable support frame 410 providing shape and support to the sealing membrane 405 along at least a portion of the sealing membrane's 405 periphery. In other words, the sealing membrane 405 is at least partially supported by the support frame 410. A particular difference between the VCD embodiments shown in FIG. 1 and FIGS. 4A and 4B is that the embodiment of the VCD 400 of FIGS. 4A and 4B includes at least one securing element 425 coupled to a tether 420. The securing element 425 is configured to engage an access channel 20 formed in a tissue 25 adjacent the puncture site 15 to prevent intraluminal migration of the VCD 400.

The support frame 410, and thus generally the VCD 400, is configured to expand from a collapsed configuration into an expanded configuration within the vessel 10, as is shown in FIGS. 4A and 4B, respectively. Upon expanding the support frame 410, the VCD 400 is configured to intraluminally position the sealing membrane 405 against the puncture site 15 to at least partially seal the puncture site 15. In some embodiments, the sealing membrane 405 and the support frame 410, and thus generally the VCD 400, may be formed in any shape configured for rolling and unrolling along a longitudinal axis generally aligned with and extending along the length of the lumen of the vessel 10 when implanted. Specifically, the support frame 410 may be formed in a shape configured for rolling into the collapsed configuration and unrolling into the expanded configuration. The expansion of the VCD 400 thus may be in a radial direction i.e., perpendicular to the longitudinal axis, within the lumen of the vessel 10. For example, as is shown, the VCD 400 may have a simple form that is similar in configuration to a sheet that can roll or unroll. However, the VCD 400 may have any other shape that can be collapsed and then expanded within a vessel to promote securement of the VCD 400 therein.

According to the embodiment shown in FIGS. 4A and 4B, the VCD 400 also includes a cross-member support 415 extending across at least a portion of the sealing membrane 405. The cross-member support 415, due to its relative rigidity and/or tension provided by the peripheral support frame 410, provides structural and shape support to the sealing membrane 405. In some embodiments, the cross-member support 415 is more rigid than the sealing membrane 405. Upon expanding the support frame 410, the cross-member support 415 is configured to maintain the sealing membrane 405 against the puncture site 15. In other words, the cross-member support 415 supports the sealing membrane 405 to avoid sagging where the sealing membrane 405 bridges the puncture site 15, thus improving the seal created therebetween. In some embodiments, the cross-member support 415 extends between opposite sides of the support frame 410 and supports the sealing membrane 405 at or near a center of the sealing membrane 405 to avoid sagging at the puncture site 15. The cross-member support 415 also is configured to increase longitudinal rigidity of the VCD 400 during deployment into the vessel 10. In this manner, the cross-member support 415 may provide the longitudinal rigidity necessary for rolling the VCD 400 along the longitudinal axis and maintaining the VCD 400 in the collapsed configuration for deployment. In such embodiments, the VCD 400 may be configured for rolling and unrolling along a longitudinal axis defined by the cross-member support 415.

In certain embodiments, the cross-member support 415 is formed separately from and attached to the support frame 410. The cross-member support 415 may be attached to opposite sides of the support frame 410. In some embodiments, the cross-member support 415 extends over the sealing membrane 405 and is configured to be positioned between the sealing membrane 405 and the wall of the vessel 10. In other embodiments, the cross-member support 415 extends beneath the sealing membrane 405 and is configured to be positioned between the sealing membrane 405 and a flow of blood through the vessel 10. The cross-member support 415 may be in the form of a flexible wire. In some embodiments, the flexible wire may be formed of a surgical suture material. In some embodiments, the flexible wire may be formed of a biodegradable material. Examples of suitable materials of construction of the flexible wire include those described above with respect to VCD 200.

As is shown in the embodiment of FIGS. 4A and 4B, the VCD 400 further includes a tether, positioning tab, or anchoring tab 420 extending from the sealing membrane 405, the support frame 410, and/or the cross-member support 415. Specifically, the tether 420 is attached to at least one of the sealing membrane 405, the support frame 410, and/or the cross-member support 415, according to certain embodiments. In some embodiments, as is shown, the tether 420 is attached to the cross-member support 415 at a securing point 422. The securing point 422 may be at about the center of the longitudinal axis of the cross-member support 415 and/or at about the center of the longitudinal axis of the VCD 400. Alternatively, the securing point 422 may be proximal or distal to the center of the longitudinal axis of the cross-member support 415 and/or the center of the longitudinal axis of the VCD 400. In a preferred embodiment, the securing point 422 is between 5 mm distal and 5 mm proximal to the center of the longitudinal axis of the VCD 400. Upon deployment of the VCD 400 within the vessel 10, the tether 420 extends out of and away from the puncture site 15. In this manner, the tether 420 may be pulled through and away from the puncture site 15 to position the sealing membrane 405 and the support frame 410 against an inner surface of the wall of the vessel 10 about the puncture site 15. Further, the tether 420 may facilitate intraluminal positioning or centering of the VCD 400 across the puncture site 15. According to some embodiments, upon positioning the VCD 400 within the vessel 10, the free end portion of the tether 420 may be affixed to the patient in a like manner as the tether 120 described above with reference to FIG. 1.

As discussed above, the support frame 410 is configured to expand from a collapsed configuration into an expanded configuration within the vessel 10. Specifically, the support frame 410 may be configured to expand from the collapsed configuration having a first radius of curvature into the expanded configuration having a second radius of curvature greater than the first radius of curvature. In some embodiments, the support frame 410 is configured to expand into the expanded configuration having a radius of curvature greater than a radius of curvature of the vessel 10. In some embodiments, the support frame 410 is formed of a self-expandable or pre-shaped material having a pre-shaped expanded configuration, such that the support frame 410 tends to assume the pre-shaped expanded configuration absent the application of external forces to the support frame 410. In this manner, the support frame 410 may be configured to self-expand from the collapsed configuration into the pre-shaped expanded configuration within the vessel 10 upon deployment or release of the VCD 400 from a containment mechanism (and consequent release of a compressive load holding the VCD 400 in the collapsed configuration). Examples of suitable materials of construction of the support frame 410 include those described above with respect to VCD 200. As is shown in FIGS. 4A and 4B, the support frame 410 is configured for rolling into the collapsed configuration and unrolling into the expanded configuration.

It has been discovered that in some instances following insertion of a VCD into the vessel 10, migration of the VCD a few millimeters proximally (i.e. in an upward direction toward the head and opposite the direction of blood flow) may occur when the physician compresses the puncture site 15 to control the bleeding until thrombus is formed between the sealing membrane of the VCD and the wall of the vessel to seal all leakage channels, if/when the physician fails to keep sufficient tension on the tether connected to the VCD while compressing the puncture site 15—even for a few seconds. The VCD 400 and related delivery systems solve this problem by allowing the VCD 400 to be secured immediately after insertion so as not to be dependent solely on the physician to maintain the tension on the tether or to affix the tether to the patient as described above with reference to FIG. 1.

According to the embodiment shown in FIGS. 4A and 4B, the VCD 400 further includes at least one securing element 425 coupled to the tether 420. The securing element 425 is configured to engage the access channel 20 formed in the tissue 25 adjacent the puncture site 15 to prevent intraluminal migration of the VCD 400. In some embodiments, the securing element 425 is configured to expand from a collapsed configuration for delivery into an expanded configuration for engaging the access channel 20.

As is shown in FIGS. 4A and 4B, the VCD 400 may be inserted into the vessel 10 via a delivery system 440. The delivery system 440 may include a sheath 442, a loading tube 444, and a push rod 446. The VCD 400 may be contained within the sheath 442 prior to delivery into the vessel 10, and the securing element 425 may be in the collapsed configuration when contained within the sheath 442. The VCD 400 then may be deployed from the sheath 442 and into the vessel 10, as is shown in FIG. 4A. Upon deployment of the VCD 400, the securing element 425 may be allowed to expand into the expanded configuration within the vessel 10. The tether 420 then may be pulled through and away from the puncture site 15 to position the sealing membrane 405 and the support frame 410 against the inner surface of the wall of the vessel 10 about the puncture site 15. In doing so, the securing element 425 may be pulled into the access channel 20. The securing element 425 may be configured to allow significantly smooth pulling of the tether 420. Specifically, as the securing element 425 is pulled into the access channel 20, the securing element 425 may collapse slightly (e.g., elastically deform) to adjust to the size of the puncture site 15 and/or the size of the access channel 20. FIG. 4B illustrates the securing element 425 in the expanded configuration and engaging the access channel 20 after delivery of the VCD 400. Due to the pulling forces applied to the tether 420 to position the sealing membrane 405 and the support frame 410 against the inner surface of the wall of the vessel 10, the tether 420 is under tension. In a preferred embodiment, the tension is effective to prevent migration of the VCD 400 within the vessel. Moreover, if a force is applied to the VCD 400 which might tend to cause the VCD 400 to migrate within the vessel 10, then the tether 420 will pull the securing element 425 toward puncture site 15, which will impale the edges of securing element 425 into the wall of the access channel 20. A longitudinal movement of the VCD 400 will pull the tether 420, which will increase the force applied by the securing element 425 on the wall of the access channel 20, improving the grip of the securing element 425. As a result, the securing element 425 will securely engage the access channel 20 and advantageously prevent any significant movement (i.e., unwanted migration) of the VCD 400.

FIGS. 4C-4E illustrate an alternative embodiment of delivering the VCD 400 into the vessel 10 via the delivery system 440 and securing the securing element 425 within the access channel 20. According to this embodiment, the VCD 400 may be contained within the sheath 442 prior to delivery into the vessel 10, and the securing element 425 may be in the collapsed configuration when contained within the sheath 442. The sealing membrane 405 and the support frame 410 then may be deployed from the sheath 442 and into the vessel 10, as is shown in FIG. 4C. Upon deployment of the sealing membrane 405 and the support frame 410, the securing element 425 remains contained within the delivery system 440, specifically the push rod 446, in the collapsed configuration. The tether 420 and the push rod 446 then may be pulled through and away from the puncture site 15 to position the sealing membrane 405 and the support frame 410 against the inner surface of the wall of the vessel 10 about the puncture site 15. In doing so, the securing element 425, while contained within the push rod 446, may be pulled into the access channel 20, as is shown in FIG. 4D. Finally, the push rod 446 may be removed from the access channel 20, allowing the securing element 425 to expand into the expanded configuration and engage the access channel 20. FIG. 4E illustrates the securing element 425 in the expanded configuration and engaging the access channel 20 after delivery of the VCD 400. Due to the pulling forces applied to the tether 420 to position the sealing membrane 405 and the support frame 410 against the inner surface of the wall of the vessel 10, the tether 420 is under tension. In a preferred embodiment, the tension is effective to prevent migration of the VCD 400 within the vessel. As a result, the securing element 425 will securely engage the access channel 20 and prevent any significant movement of VCD 400. According to this embodiment of delivering the VCD 400, the securing element 425 expands only within the access channel 20, which reduces the risk of the securing element 425 being caught in or causing injury to the vessel 10.

FIGS. 4F-4I illustrate several example embodiments of the securing element 425 in the expanded configuration. Other embodiments of the securing element 425 are also envisioned. In some embodiments, as is shown in FIGS. 4F and 4G, the securing element 425 is formed as a ribbon. The ribbon may be flexible such that the securing element 425 may expand from the collapsed configuration for delivery into the expanded configuration for engaging the access channel 20. In some embodiments, as is shown in FIG. 4F, the ribbon is pre-shaped to define a flat shape when in the expanded configuration. In other embodiments, as is shown in FIG. 4G, the ribbon is pre-shaped to define an angled shape when in the expanded configuration. For example, the ribbon may define an angle 427 between opposite ends of the ribbon. The angle 427 may range from between 90° to 180°, and preferably between 130° to 170°. The pre-shaping of the angled shape of the ribbon may reduce resistance to movement of the securing element 425 into the access channel 20 during pull back of the tether 420, while increasing resistance to movement of the securing element 425 toward the puncture site 15 due to migration of the VCD 400 after implantation.

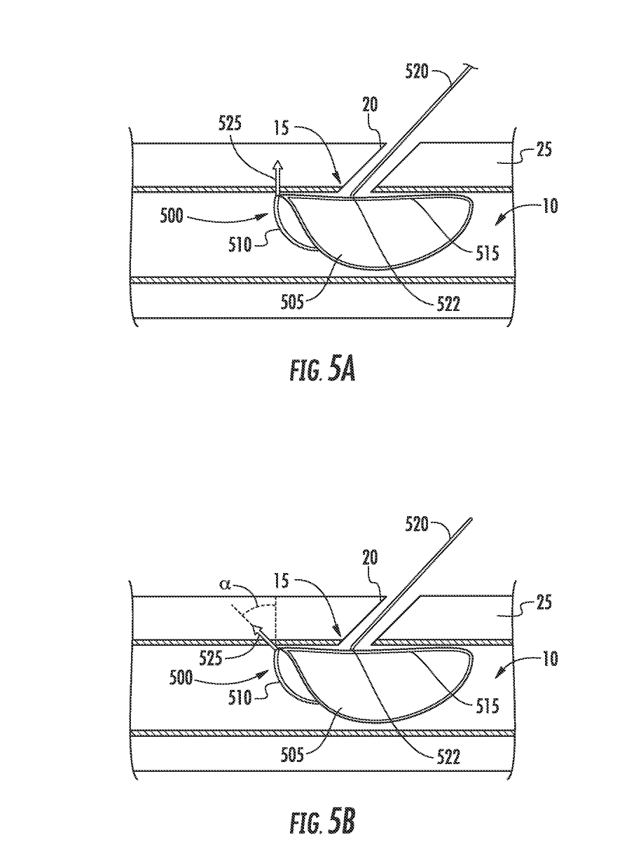

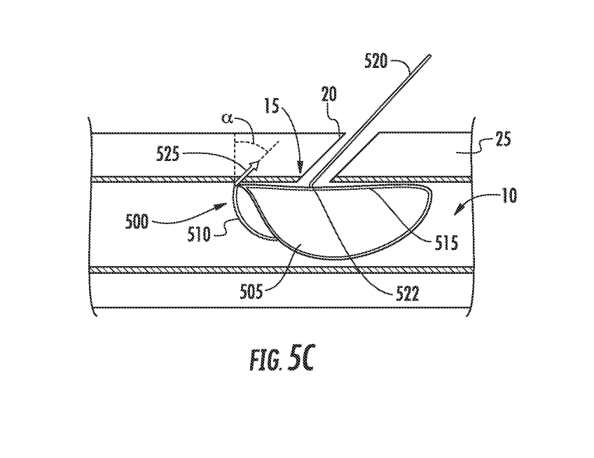

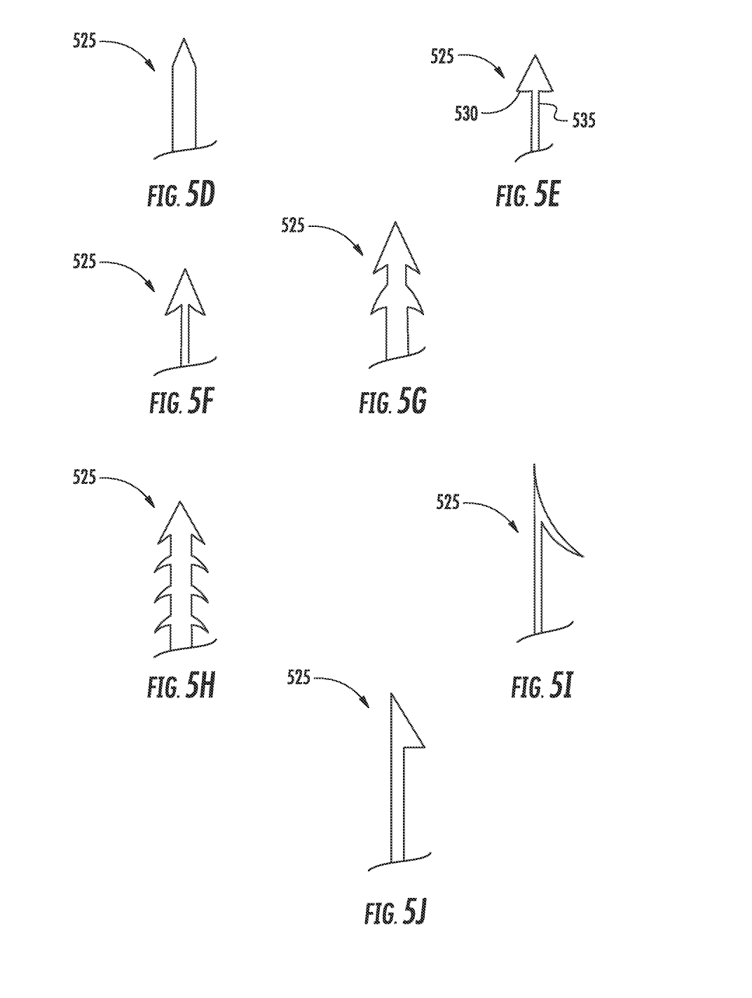

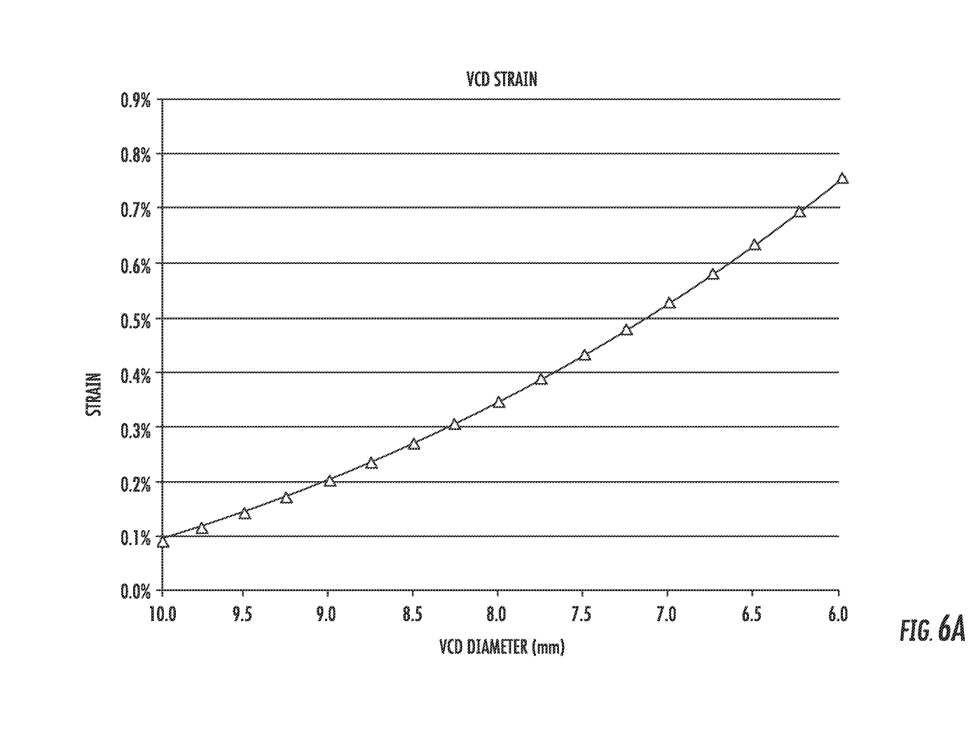

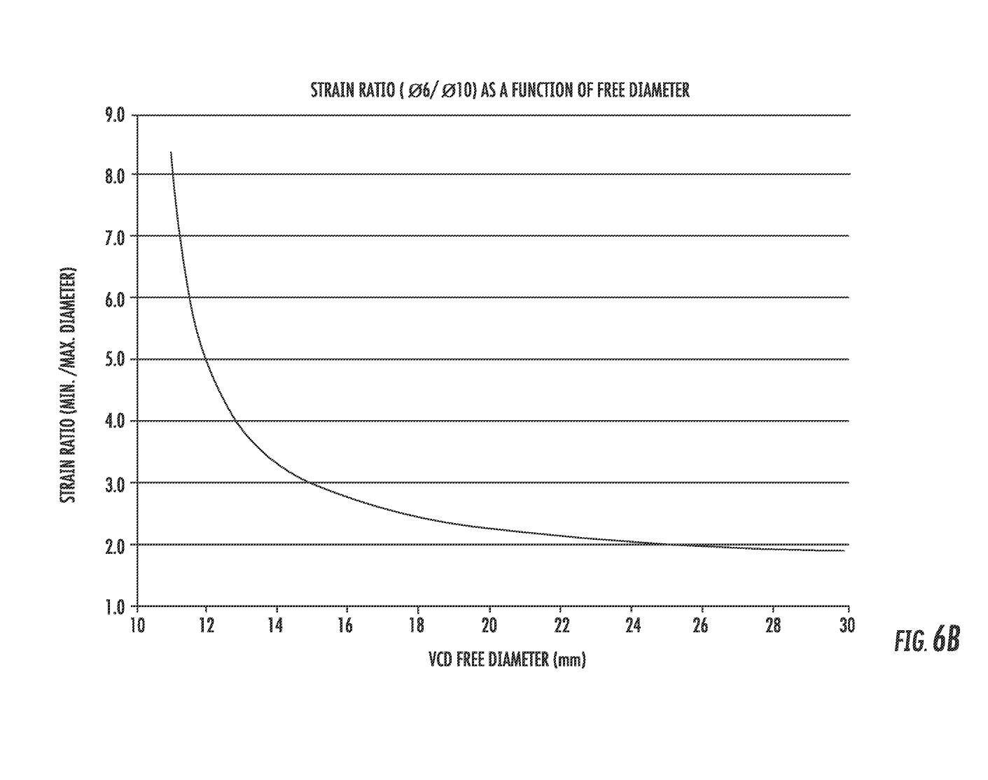

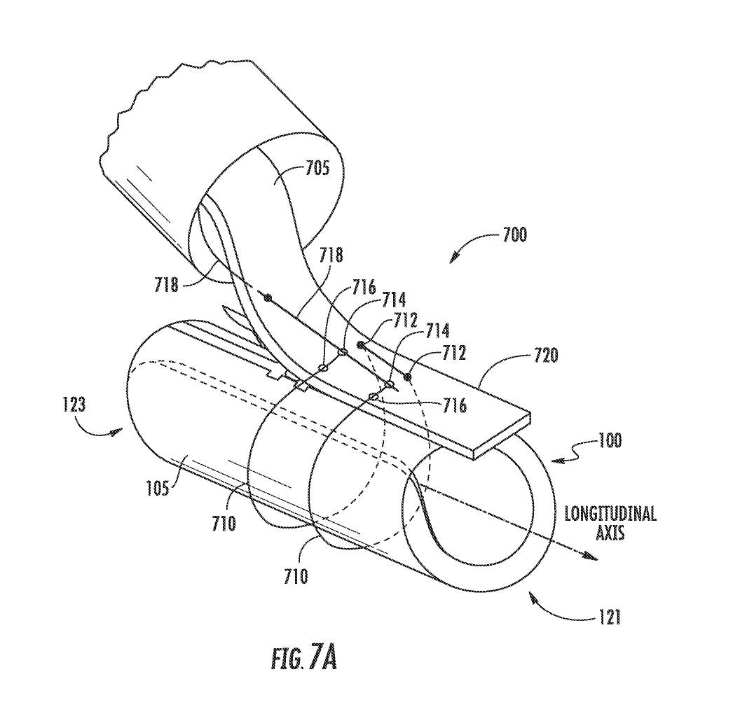

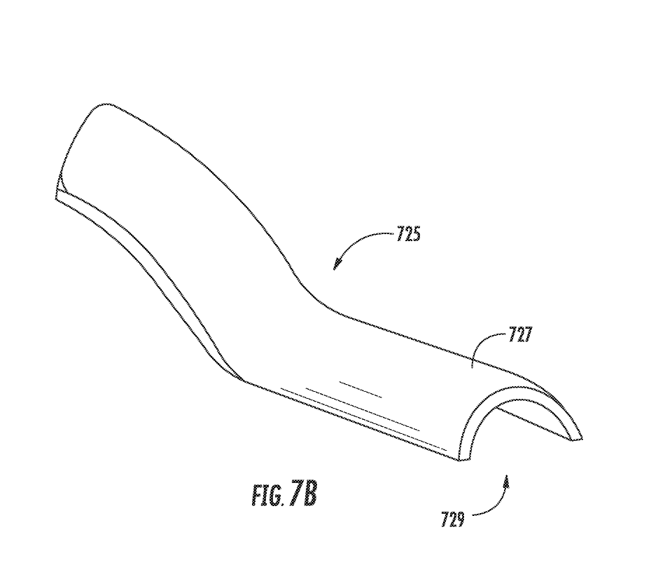

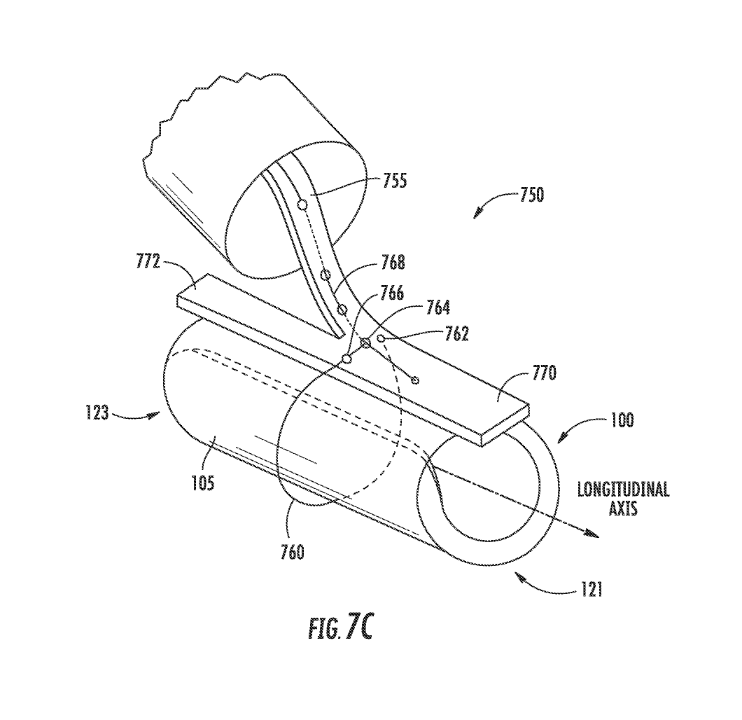

In some embodiments, as is shown in FIG. 4F, the securing element 425 includes one or more flat edges 428 configured to engage the walls of the access channel 20. In other embodiments, as is shown in FIG. 4G, the securing element 425 includes one or more sharp edges 429, such as a barb, hook, point, or spike, configured to engage the walls of the access channel 20. For example, the sharp edges 429 may include any of the barbs, hooks, points, or spikes illustrated in FIGS. 5D-5J, or a combination of these features, to improve fixation of the securing element 425 within the access channel 20.

The securing element 425 may be dimensioned to securely engage the walls of the access channel 20 when in the expanded configuration. For example, the securing element 425 may be dimensioned to securely engage the walls of an access channel 20 formed by an 18Fr introducer sheath having an outer diameter of about 7.5 mm. In many cases, as soon as the sheath is removed, the access channel 20 will recoil, significantly decreasing its diameter. The length of the securing element 425 generally may be at least similar to the actual diameter of the access channel 20, and preferably may be longer in order to allow the securing element 425 to engage the walls of the access channel 20 by flexing and to apply force on the walls of the access channel 20. For an access channel 20 formed by an 18Fr introducer sheath, the length of the securing element 425 length generally may be between about 3 mm and about 12 mm, and more preferably between about 5 mm and about 9 mm. In some embodiments, the thickness of the securing element 425 is between about 0.02 mm and about 1 mm, and in some other embodiments, between about 0.05 mm and 0.5 mm. In some embodiments, the width of the securing element 425 is between about 0.05 mm and about 5 mm, and in some other embodiments, between about 0.2 mm and about 3 mm. In yet another embodiment, the width of the securing element 425 is between about 0.5 mm and about 2 mm.

The securing element 425 may be formed of essentially any elastic or super elastic material. Examples of suitable materials include biocompatible metallic materials (e.g., stainless steel, cobalt alloys, nickel-titanium or similar metals and alloys) and biocompatible polymeric materials (e.g., PEEK (polyethylether ketone), fluorocarbon polymers, polyamides, polyimides, polyethylenes, polypropylenes or similar polymers and copolymers). In a preferred embodiment, the securing element 425 is formed of a biodegradable material (e.g., PLLA, PDLA, PGA, PLGA, PDS, PCL, PGA-TMC, polygluconate, polylactic acid-polyethylene oxide).

The securing element 425 may include one or more fixation means for coupling the tether 420 to the securing element 425 to prevent its movement relative to the puncture site 15, at least its movement away from the puncture site 15 and toward the patient's outer skin surface. In some embodiments, the securing element 425 is fixed to tether 420 to prevent any relative movement between them. The fixation means for coupling the tether 420 to the securing element 425 to may include at least one hole, slot, groove, or similar feature defined in the securing element 425 and in which the tether 420 can be engaged. The tether 420 may be sutured, glued, welded, braided, or mechanically fastened to the securing element 425.

In some embodiments, as is shown in FIGS. 4H and 4I, the securing element 425 includes a plurality of barbs 430 extending outward from a center of the securing element 425. According to the embodiment of FIG. 4H, the securing element 425 includes three barbs 430 defining an angle 431 between adjacent barbs 430. According to the embodiment of FIG. 4I, the securing element 425 includes four barbs 430 defining an angle 432 between adjacent barbs 430. The tips of the barbs may include one or more flat edges 428 or sharp edges 429 configured to engage the walls of the access channel 20. The materials, dimensions, angles, and edges of the embodiments of the securing element 425 of FIGS. 4H and 4I may be similar to those discussed above with respect to the embodiments of FIGS. 4F and 4G.

FIGS. 4J and 4K illustrate one embodiment of a VCD 450, similar to the VCD 400 illustrated in and described with reference to FIGS. 4A and 4B, although certain differences in structure and function are described herein below. According to this embodiment, the VCD 450 includes a sealing membrane 455 and an expandable support frame 460 providing shape and support to the sealing membrane 455 along at least a portion of the sealing membrane's 455 periphery. In other words, the sealing membrane 455 is at least partially supported by the support frame 460. A particular difference between the VCD embodiments shown in FIGS. 4A and 4B and FIGS. 4J and 4K is that the embodiment of the VCD 450 of FIGS. 4J and 4K includes at least one securing element 475 configured to rotate from an upright position to an angled position for engaging the walls of the access channel 20 to prevent intraluminal migration of the VCD 450.

The support frame 460, and thus generally the VCD 450, is configured to expand from a collapsed configuration into an expanded configuration within the vessel 10. Upon expanding the support frame 460, the VCD 450 is configured to intraluminally position the sealing membrane 455 against the puncture site 15 to at least partially seal the puncture site 15. In some embodiments, the sealing membrane 455 and the support frame 460, and thus generally the VCD 450, is formed in any shape configured for rolling and unrolling along a longitudinal axis generally aligned with and extending along the length of the lumen of the vessel 10 when implanted. Specifically, the support frame 460 may be formed in a shape configured for rolling into the collapsed configuration and unrolling into the expanded configuration. The expansion of the VCD 450 thus may be in a radial direction i.e., perpendicular to the longitudinal axis, within the lumen of the vessel 10. For example, as is shown, the VCD 450 may have a simple form that is similar in configuration to a sheet that can roll or unroll. However, the VCD 450 may have any other shape that can be collapsed and then expanded within a vessel to promote securement of the VCD 450 therein.

According to the embodiment shown in FIGS. 4J and 4K, the VCD 450 also includes a cross-member support 465 extending across at least a portion of the sealing membrane 455. The cross-member support 465, due to its relative rigidity and/or tension provided by the peripheral support frame 460, provides structural and shape support to the sealing membrane 455. In some embodiments, the cross-member support 465 is more rigid than the sealing membrane 455. Upon expanding the support frame 460, the cross-member support 465 is configured to maintain the sealing membrane 455 against the puncture site 15. In other words, the cross-member support 465 supports the sealing membrane 455 to avoid sagging where the sealing membrane 455 bridges the puncture site 15, thus improving the seal created therebetween. In some embodiments, the cross-member support 465 extends between opposite sides of the support frame 460 and supports the sealing membrane 455 at or near a center of the sealing membrane 455 to avoid sagging at the puncture site 15. The cross-member support 465 also is configured to increase longitudinal rigidity of the VCD 450 during deployment into the vessel 10. In this manner, the cross-member support 465 may provide the longitudinal rigidity necessary for rolling the VCD 450 along the longitudinal axis and maintaining the VCD 450 in the collapsed configuration for deployment. In such embodiments, the VCD 450 may be configured for rolling and unrolling along a longitudinal axis defined by the cross-member support 465.

In certain embodiments, the cross-member support 465 is formed separately from and attached to the support frame 460. The cross-member support 465 may be attached to opposite sides of the support frame 460. In some embodiments, the cross-member support 465 extends over the sealing membrane 455 and is configured to be positioned between the sealing membrane 455 and the wall of the vessel 10. In other embodiments, the cross-member support 465 extends beneath the sealing membrane 455 and is configured to be positioned between the sealing membrane 455 and a flow of blood through the vessel 10. The cross-member support 455 may be in the form of a flexible wire. In some embodiments, the flexible wire may be formed of a surgical suture material. In some embodiments, the flexible wire may be formed of a biodegradable material. Examples of suitable materials of construction of the flexible wire include those described above with respect to VCD 200.

As is shown in the embodiment of FIGS. 4J and 4K, the VCD 450 further includes a tether, positioning tab, or anchoring tab 470 extending from the sealing membrane 455, the support frame 460, and/or the cross-member support 465. Specifically, the tether 470 is attached to at least one of the sealing membrane 455, the support frame 460, and/or the cross-member support 465, according to certain embodiments. In some embodiments, as is shown, the tether 470 is attached to the cross-member support 465 at a securing point 472. The securing point 472 may be at about the center of the longitudinal axis of the cross-member support 465 and/or at about the center of the longitudinal axis of the VCD 450. Alternatively, the securing point 472 may be proximal or distal to the center of the longitudinal axis of the cross-member support 465 and/or the center of the longitudinal axis of the VCD 450. In a preferred embodiment, the securing point 472 is between 5 mm distal and 5 mm proximal to the center of the longitudinal axis of the VCD 450. Upon deployment of the VCD 450 within the vessel 10, the tether 470 extends out of and away from the puncture site 15. In this manner, the tether 470 may be pulled through and away from the puncture site 15 to position the sealing membrane 455 and the support frame 460 against an inner surface of the wall of the vessel 10 about the puncture site 15. Further, the tether 470 may facilitate intraluminal positioning or centering of the VCD 450 across the puncture site 15. According to some embodiments, upon positioning the VCD 450 within the vessel 10, the free end portion of the tether 470 may be affixed to the patient in a like manner as the tether 120 described above with reference to FIG. 1.

As discussed above, the support frame 460 is configured to expand from a collapsed configuration into an expanded configuration within the vessel 10. Specifically, the support frame 460 may be configured to expand from the collapsed configuration having a first radius of curvature into the expanded configuration having a second radius of curvature greater than the first radius of curvature. In some embodiments, the support frame 460 is configured to expand into the expanded configuration having a radius of curvature greater than a radius of curvature of the vessel 10. In some embodiments, the support frame 460 is formed of a self-expandable or pre-shaped material having a pre-shaped expanded configuration, such that the support frame 460 tends to assume the pre-shaped expanded configuration absent the application of external forces to the support frame 460. In this manner, the support frame 460 may be configured to self-expand from the collapsed configuration into the pre-shaped expanded configuration within the vessel 10 upon deployment or release of the VCD 450 from a containment mechanism (and consequent release of a compressive load holding the VCD 450 in the collapsed configuration). Examples of suitable materials of construction of the support frame 460 include those described above with respect to VCD 200. As is shown in FIGS. 4J and 4K, the support frame 460 is configured for rolling into the collapsed configuration and unrolling into the expanded configuration.

According to the embodiment shown in FIGS. 4J and 4K, the VCD 450 further includes at least one securing element 475 coupled to the tether 470. The securing element 475 is configured to engage the walls of the access channel 20 formed in the tissue 25 adjacent the puncture site 15 to prevent intraluminal migration of the VCD 450. As is shown, the securing element 475 is elongated and configured to rotate from an upright position to an angled position for engaging the walls of the access channel 20. In some embodiments, the securing element 475 is formed as a relatively rigid rod or pin, having a total length larger than the diameter of the access channel 20 after removal of the introducer sheath or delivery system used for performing a minimally invasive procedure. In the illustrated embodiment, a proximal end 477 of the securing element 475 is coupled to the tether 470, and a distal end 479 of the securing element 475 is coupled to a delivery tether or anchoring tab 494 of a delivery system 490.

As is shown in FIGS. 4J and 4K, the VCD 450 may be inserted into the vessel 10 via the delivery system 490. The delivery system 490 may include a delivery sheath 492 and a delivery tether 494. The distal end 479 of the securing element 475 may be coupled to the delivery tether 494. The VCD 450 may be contained within the sheath 492 prior to delivery into the vessel 10, and the securing element 475 may be in the upright position when contained within the sheath 492. When in the upright position, the proximal end 477 of the securing element 475 points away from the puncture site 15 and the distal end 479 of the securing element 475 points toward the puncture site 15, such that the longitudinal axis of the securing element 475 is about parallel to the axis of the access channel 20. The sealing membrane 455 and the support frame 460 may be deployed from the sheath 492 and into the vessel 10, as is shown in FIG. 4J. Upon deployment of the sealing membrane 455 and the support frame 460, the securing element 475 remains contained within the delivery system 490, specifically the sheath 492, in the upright position. The sealing membrane 455 and the support frame 460 then may expand and be positioned against the inner surface of the wall of the vessel 10 about the puncture site 15 while the delivery tether 494 is pulled away from the puncture site 15, resulting in tension on the delivery tether 494. Next, the sheath 492 may be removed from the access channel 20 while tension is maintained on the delivery tether 494.

As the physician or other user continues to pull the delivery tether 494 away from the puncture site 15, the distal end 479 of the securing element 475 is pulled away from the puncture site 15 while the proximal end 477 of the securing element 475 retains its distance from the puncture site 15, due to the tether 470. As a result, the securing element 475 will rotate into the angled position and engage the walls of the access channel 20, as is shown in FIG. 4K. Because the total length of the securing element 475 is significantly longer than the diameter of the access channel 20, the proximal end 477 and the distal end 479 of the securing element 475 engage the walls of the access channel 20. Due to the pulling forces applied to the tether 470 and the angled position of the securing element 475 engaging the walls of the access channel 20, the tether 470 is under tension. In a preferred embodiment, the tension is effective to prevent migration of the VCD 450 within the vessel. Moreover, if a force is applied to the VCD 450 which might tend to cause the VCD 450 to migrate within the vessel 10, then the tether 470 will attempt to further rotate the securing element 475, which will impale the proximal end 477 and the distal end 479 of securing element 475 further into the walls of the access channel 20. A longitudinal movement of the VCD 450 will pull the tether 470, which will increase the force applied by the securing element 475 on the wall of the access channel 20, improving the grip of the securing element 475. As a result, the securing element 475 will securely engage the access channel 20 and advantageously prevent any significant movement (i.e., unwanted migration) of the VCD 450.

In some embodiments, the tether 470 is coupled to the proximal end 477 and the distal end 479 of the securing element 475, and the delivery tether 494 is coupled to the proximal end 477 and the distal end 479 of the securing element 475, as is shown by dashed lines in FIGS. 4J and 4K. In this manner, the multiple coupling points of the tether 470 and the delivery tether 494 may limit a range of rotation of the securing element 475. For example, the tether 470 and the delivery tether 494 may prevent the securing element 475 from rotating more than 90° from the upright position into the angled position to ensure that the securing element 475 remains securely engaged with the walls of the access channel 20.

FIGS. 4L and 4M illustrate example embodiments of the securing element 475 formed as a relatively rigid rod or pin. Other embodiments of the securing element 475 are also envisioned. The securing element 475 may have an elongated, cylindrical shape. Alternatively, the securing element 475 may have an elongated, rectangular shape. In some embodiments, as is shown in FIG. 4L, the proximal end 477 and the distal end 479 of the securing element 475 have a conical shape configured to engage the walls of the access channel 20. In other embodiments, as is shown in FIG. 4M, the proximal end 477 and the distal end 479 of the securing element 475 include a planar bevel configured to engage the walls of the access channel 20. The planar bevel may for example be formed by cutting the cylindrical body of the securing element 475 an angle of between 75° and 15° relative to the longitudinal axis of the securing element 475. In still other embodiments, the proximal end 477 and the distal end 479 include multiple angled surfaces and/or edges configured to engage the walls of the access channel 20. For example, the proximal end 477 and the distal end 479 may include any of the barbs, hooks, points, or spikes illustrated in FIGS. 5D-5J, or a combination of these features, to improve fixation of the securing element 475 within the access channel 20.

As discussed above, the securing element 475 preferably has a total length larger than the diameter of the access channel 20 after removal of the introducer sheath or delivery system. For example, for an 18 Fr introducer sheath, the total length of the securing element 475 may be between about 5 mm and about 12 mm, or in some other embodiments, between about 6 mm and about 10 mm. The diameter of the securing element 475 may be between about 0.1 mm and about 3 mm, or in some other embodiments, between about 0.5 mm and about 2 mm. The securing element 475 may include fixing means for coupling the tether 470 and the delivery tether 494 to the securing element 475. The fixing means may include one or more holes, hooks, slots, grooves, or the like, according to various embodiments of the securing element 475. The tether 470 and the delivery tether 494 may be coupled to the fixing means in a number of ways, including suturing, gluing, braiding, mechanical fasteners, or essentially any other coupling technique known in the art. In some embodiments, the delivery tether 494 is detachably coupled to the securing element 475. In this manner, the delivery tether 494 may be detached from the securing element 475 and removed after the securing element 475 securely engages the walls of the access channel 20. In such embodiments, the delivery tether 494 may be detachable by a friction, peeling, or tearing connection, which also limits the pulling force that may be applied by the delivery tether 494 to the securing element 475.

The securing element 475 may be formed of essentially any relatively rigid material. Examples of suitable materials include biocompatible metallic materials (e.g., stainless steel, cobalt alloys, nickel-titanium or similar metals and alloys) and biocompatible polymeric materials (e.g., PEEK (polyethylether ketone), fluorocarbon polymers, polyamides, polyimides, polyethylenes, polypropylenes or similar polymers and copolymers). In one embodiment, the securing element 475 is formed of a biodegradable material (e.g., PLLA, PDLA, PGA, PLGA, PDS, PCL, PGA-TMC, polygluconate, polylactic acid-polyethylene oxide).

FIG. 4N illustrates an alternative embodiment of a VCD including a securing element configured to temporarily engage the walls of the access channel 20 to prevent intraluminal migration of the VCD until hemostasis is obtained at the puncture site 15. According to the embodiments of the VCD 400 and the VCD 450 and related methods of delivery described above, the securing element may be implanted and remain within the access channel 20 after hemostasis is obtained at the puncture site 15. In contrast, according to the alternative embodiment of the VCD of FIG. 4N, the securing element 425 is detached from the VCD and removed from the access channel 20 after hemostasis is obtained at the puncture site 15. Although the structure and function of the alternative embodiment may be applied to the VCD 400 or the VCD 450 described above, FIG. 4N illustrates the alternative embodiment as applied to the VCD 400.

According to this embodiment, the securing element 425 is detachably coupled to the tether 420 of the VCD 400. As is shown in FIG. 4N, the tether 420 includes a loop 426 threaded over the securing element 425 to detachably couple the securing element 425 to the tether 420. Other detachable fixation means are contemplated. The securing element 425 also is coupled to a delivery tether 448 of the delivery system 440. The delivery tether 448 may be configured to extend through the access channel 20 and out of the patient's body for manipulation during delivery of the VCD 400. The delivery tether 448 may be flexible (e.g. suture wire), semi-rigid (e.g. metal wire or ribbon) or rigid, according to various embodiments.

Delivery of the VCD 400 may be performed as illustrated and described above with reference to FIGS. 4A and 4B. After delivery of the VCD 400 into the vessel 10, the securing element 425 is in the expanded configuration and engages the walls of the access channel 20, and the proximal end of the delivery tether 448 extends out of the patient's body, as is shown in FIG. 4N. After hemostasis is obtained at the puncture site 15, and the additional fixation of the securing element 425 is no longer required, the securing element 425 is detached from the tether 420 and removed from the access channel 20. Specifically, the proximal end of the delivery tether 448 is pulled such that the securing element 425 is removed from the loop 426 of the tether 420. In other words, upon pulling the delivery tether 448, the loop 426 slides off of the securing element 425. According to some embodiments, before detaching the securing element 425 from the tether 420, the tether 420 is fixed to the patient in a like manner as tether 120 described above with reference to FIG. 1. In some embodiments, the securing element 425 is removed within minutes after delivery of the VCD 400, while in some other embodiments, the securing element 425 is removed within hours of delivery. In yet other embodiments, the securing element 425 is removed within about 1 to 4 days after delivery.

FIG. 5A illustrates one embodiment of a VCD 500, similar to the VCD 100 illustrated in and described with reference to FIG. 1, although certain differences in structure and function are described herein below. According to this embodiment, the VCD 500 includes a sealing membrane 505 and an expandable support frame 510 providing shape and support to the sealing membrane 505 along at least a portion of the sealing membrane's 505 periphery. In other words, the sealing membrane 505 is at least partially supported by the support frame 510. A particular difference between the VCD embodiments shown in FIG. 1 and FIG. 5A is that the embodiment of the VCD 500 of FIG. 5A includes at least one fixation element 525 extending from the support frame 510. The securing element 525 is configured to penetrate the wall of the vessel 10 adjacent the puncture site 15 to prevent intraluminal migration of the VCD 500.

The support frame 510, and thus generally the VCD 500, is configured to expand from a collapsed configuration into an expanded configuration within the vessel 10. Upon expanding the support frame 510, the VCD 500 is configured to intraluminally position the sealing membrane 505 against the puncture site 15 to at least partially seal the puncture site 15, as is shown in FIG. 5A. In some embodiments, the sealing membrane 505 and the support frame 510, and thus generally the VCD 500, is formed in any shape configured for rolling and unrolling along a longitudinal axis generally aligned with and extending along the length of the lumen of the vessel 10 when implanted. The expansion of the VCD 500 thus may be in a radial direction i.e., perpendicular to the longitudinal axis, within the lumen of the vessel 10. Specifically, the support frame 510 may be formed in a shape configured for rolling into the collapsed configuration and unrolling into the expanded configuration. For example, as is shown, the VCD 500 may have a simple form that is similar in configuration to a sheet that can roll or unroll. However, the VCD 500 may have any other shape that can be collapsed and then expanded within a vessel to promote securement of the VCD 500 therein.

The VCD 500 also may include a cross-member support 515 extending across at least a portion of the sealing membrane 505. The cross-member support 515, due to its relative rigidity and/or tension provided by the peripheral support frame 510, provides structural and shape support to the sealing membrane 505. In some embodiments, the cross-member support 515 is more rigid than the sealing membrane 505. Upon expanding the support frame 510, the cross-member support 515 may be configured to maintain the sealing membrane 505 against the puncture site 15. In other words, the cross-member support 515 may support the sealing membrane 505 to avoid sagging where the sealing membrane 505 bridges the puncture site 15, thus improving the seal created therebetween. In some embodiments, the cross-member support 515 extends between opposite sides of the support frame 510 and supports the sealing membrane 505 at or near a center of the sealing membrane 505 to avoid sagging at the puncture site 15. The cross-member support 515 also may be configured to increase longitudinal rigidity of the VCD 500 during deployment into the vessel 10. In this manner, the cross-member support 515 may provide the longitudinal rigidity necessary for rolling the VCD 500 along the longitudinal axis and maintaining the VCD 500 in the collapsed configuration for deployment. In such embodiments, the VCD 500 may be configured for rolling and unrolling along a longitudinal axis defined by the cross-member support 515.

In certain embodiments, the cross-member support 515 is formed separately from and attached to the support frame 510. The cross-member support 515 may be attached to opposite sides of the support frame 510. In some embodiments, the cross-member support 515 extends over the sealing membrane 505 and is configured to be positioned between the sealing membrane 505 and the wall of the vessel 10. In other embodiments, the cross-member support 515 extends beneath the sealing membrane 505 and is configured to be positioned between the sealing membrane 505 and a flow of blood through the vessel 10. The cross-member support 515 may be in the form of a flexible wire. In some embodiments, the flexible wire is formed of a surgical suture material. In some embodiments, the flexible wire is formed of a biodegradable material. Examples of suitable materials of construction of the flexible wire include those described above with respect to VCD 200.

As is shown in the embodiment of FIG. 5A, the VCD 500 further includes a tether, positioning tab, or anchoring tab 520 extending from the sealing membrane 505, the support frame 510, and/or the cross-member support 515. Specifically, the tether 520 is attached to at least one of the sealing membrane 505, the support frame 510, and/or the cross-member support 515, according to certain embodiments. In some embodiments, as is shown, the tether 520 is attached to the cross-member support 515 at a securing point 522. The securing point 522 may be at about the center of the longitudinal axis of the cross-member support 515 and/or at about the center of the longitudinal axis of the VCD 500. Alternatively, the securing point 522 may be proximal or distal to the center of the longitudinal axis of the cross-member support 515 and/or the center of the longitudinal axis of the VCD 500. In a preferred embodiment, the securing point 522 is between 5 mm distal and 5 mm proximal to the center of the longitudinal axis of the VCD 500. Upon deployment of the VCD 500 within the vessel 10, the tether 520 extends out of and away from the puncture site 15. In this manner, the tether 520 may be pulled through and away from the puncture site 15 to position the sealing membrane 505 and the support frame 510 against an inner surface of the wall of the vessel 10 about the puncture site 15. Further, the tether 520 may facilitate intraluminal positioning or centering of the VCD 500 across the puncture site 15. According to some embodiments, upon positioning the VCD 500 within the vessel 10, the free end portion of the tether 520 may be affixed to the patient in a like manner as the tether 120 described above with reference to FIG. 1.

As discussed above, the support frame 510 is configured to expand from a collapsed configuration into an expanded configuration within the vessel 10. Specifically, the support frame 510 may be configured to expand from the collapsed configuration having a first radius of curvature into the expanded configuration having a second radius of curvature greater than the first radius of curvature. In some embodiments, the support frame 510 is configured to expand into the expanded configuration having a radius of curvature greater than a radius of curvature of the vessel 10. In some embodiments, the support frame 510 is formed of a self-expandable or pre-shaped material having a pre-shaped expanded configuration, such that the support frame 510 tends to assume the pre-shaped expanded configuration absent the application of external forces to the support frame 510. In this manner, the support frame 510 may be configured to self-expand from the collapsed configuration into the pre-shaped expanded configuration within the vessel 10 upon deployment or release of the VCD 500 from a containment mechanism (and consequent release of a compressive load holding the VCD 500 in the collapsed configuration). Examples of suitable materials of construction of the support frame 510 include those described above with respect to VCD 200. As is shown in FIG. 5A, the support frame 510 is configured for rolling into the collapsed configuration and unrolling into the expanded configuration.

According to the embodiment shown in FIG. 5A, the VCD 500 further includes at least one fixation element 525 extending from the support frame 510. The fixation element 525 is configured to penetrate the wall of the vessel 10 adjacent the puncture site 15 to prevent intraluminal migration of the VCD 500. The fixation element 525 also may be configured to penetrate the tissue 25 surrounding the vessel 10 adjacent the puncture site 15. In some embodiments, the fixation element 525 is integrally formed with the support frame 510. In other embodiments, the fixation element 525 is formed separately from and attached to the support frame 510. For example, the fixation element 525 may be attached to the support frame 510 by welding, gluing, pressing, anodic bonding, press fitting, riveting, or other mechanical fastener or interlock feature. In some embodiments, as is shown in FIG. 5A, the fixation element 525 is located on a proximal end of the support frame 510. In other embodiments, the fixation element 525 is located on a distal end of the support frame 510. The fixation element 525 generally may be located on at any position along the support frame 510. In some embodiments, the VCD 500 includes a plurality of fixation elements 525 extending from the support frame 510, including at least one fixation element 525 located on a proximal end of the support frame 510 and at least one fixation element 525 located on a distal end of the support frame 510. In other embodiments, the VCD 500 includes one or more fixation elements 525 extending from any other part of the VCD 500, including but not limited to the sealing membrane 505, the cross-member support 515, or the tether 520.

As discussed above, the support frame 510 is configured to expand from a collapsed configuration into an expanded configuration within the vessel 10. In some embodiments, the fixation element 525 is configured to penetrate the wall of the vessel 10 at least partially as a result of radial expansion of the support frame 510. Additionally, in some embodiments, the fixation element 525 is configured to penetrate the wall of the vessel 10 at least partially as a result of pulling the tether 520 through the puncture site 15 after the VCD 500 is delivered into the vessel 10. Further, in some embodiments, the fixation element 525 is configured to penetrate the wall of the vessel 10 at least partially as a result of blood pressure applied on the VCD 500 after the VCD 500 is delivered into the vessel 10.

The fixation element 525 may extend from the support frame 510 at a predetermined angle relative to the support frame 510. In some embodiments, the fixation element 525 is thermally treated to maintain the predetermined angle and resist movement relative to the support frame 510. According to the embodiment shown in FIG. 5A, the fixation element 525 extends about perpendicular to the longitudinal axis of the support frame 510. In this manner, the fixation element 525 is configured to penetrate the wall of the vessel 10 at an angle about perpendicular to the wall of the vessel 10. FIG. 5B illustrates an alternative embodiment of the fixation element 525 extending proximally at a non-perpendicular angle relative to longitudinal axis of the support frame 510. In other words, the fixation element 525 extends proximally at an angle α relative to an axis perpendicular to the longitudinal axis of the support frame 510. In this manner, the fixation element 525 is configured to penetrate the wall of the vessel 10 at an angle extending toward a proximal portion of the vessel 10, such that the fixation element 525 provides better resistance to proximal migration of the VCD 500. In some embodiments, the angle α is between about 5° to 75°. FIG. 5C illustrates another alternative embodiment of the fixation element 525 extending distally at a non-perpendicular angle relative to longitudinal axis of the support frame 510. In other words, the fixation element 525 extends distally at an angle α relative to an axis perpendicular to the longitudinal axis of the support frame 510. In this manner, the fixation element 525 is configured to penetrate the wall of the vessel 10 at an angle extending toward a distal portion of the vessel 10, such that the fixation element 525 provides better resistance to distal migration of the VCD 500. Additionally, the distally extending angle of the fixation element 525 may ease penetration of the fixation element 525 into the wall of the vessel 10 due to force applied by pulling the tether 520. In some embodiments, the angle α is between about 5° to 75°.

FIGS. 5D-J illustrate example embodiments of the fixation element 525 having various shapes, including arrows, barbs, hooks, points, or spikes. Other shapes for the fixation element 525 are contemplated. In some embodiments, the fixation element 525 includes a sharp edge or projection configured to ease penetration of the wall of the vessel 10 and possibly the tissue 25 surrounding the vessel 10. Additionally, in some embodiments, the fixation element 525 includes a sharp edge or projection configured to resist removal from the wall of the vessel 10 and possibly the tissue 25 surrounding the vessel 10. In embodiments of the VCD 500 including a plurality of fixation elements 525, the fixation elements 525 may have the same shape or different shapes.

In some embodiments, the total length of the fixation element 525 is between about 0.2 mm and about 9 mm, such as between about 1 mm and about 5 mm, or between about 2 mm and about 4 mm. In embodiments of the fixation element 525 having an arrow or barb, the width of a step 530 of the arrow or barb may range between about 0.1 mm and about 2.5 mm, such as between about 0.2 mm and 1 mm. The width of a holding bar 535 of the arrow or barb may range between about 0.1 mm and about 3 mm, such as between about 0.5 mm and about 1.5 mm. The fixation element 525 may be formed from a relatively thin sheet of material, which will result in a “two-dimensional” arrow or barb, or the fixation element 525 may be made using milling, casting, 3D etching, laser cutting or the like, to form a three-dimensional arrow or barb.

The fixation element 525 may be manufactured from essentially any biocompatible metallic materials (e.g., stainless steel, cobalt alloys, nickel-titanium, gold, platinum or similar metals and alloys) or biocompatible polymeric materials (e.g., PEEK, fluorocarbon polymers, polyamides, polyimides, polyethylenes, polypropylenes or similar polymers and copolymers). In a preferred embodiment, the fixation element 525 is made of a biodegradable material (e.g., PLLA, PDLA, PGA, PLGA, PDS, PCL, PGA-TMC, polygluconate, polylactic acid-polyethylene oxide).

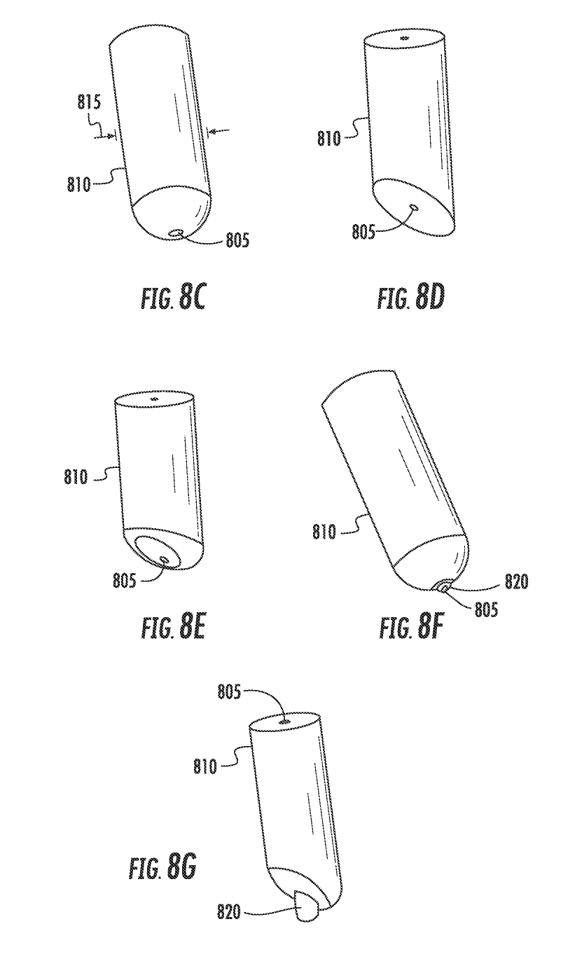

The fixation element 525 may be delivered at about its final angle relative to the support frame 510. In some embodiments, the fixation element 525, during delivery, is retained at a different angle than its final or predetermined angle, and upon removing a containment means, the fixation element 525 is elastically returned to its final or predetermined angle. In some embodiments, the fixation element 525 is covered during delivery to prevent it from injuring the wall of the vessel 10. For example, a delivery system component, such as an implant holder as described below with reference to FIG. 7A, may cover the fixation element 525 at least partially during delivery of the VCD 500. In some embodiments, the same covering means also holds the fixation element 525 in a bended state to keep the tip of the fixation element 525 away from the vessel wall.

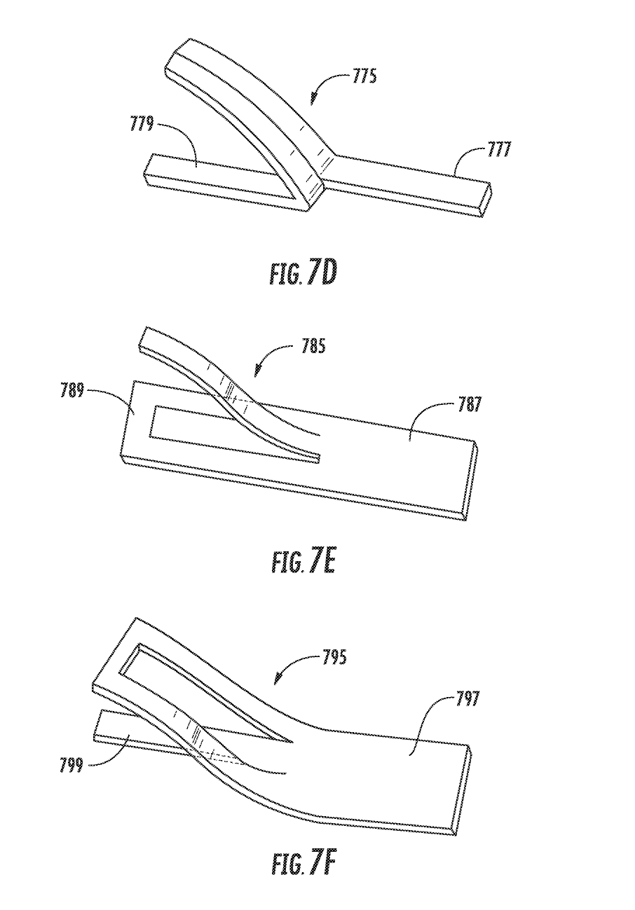

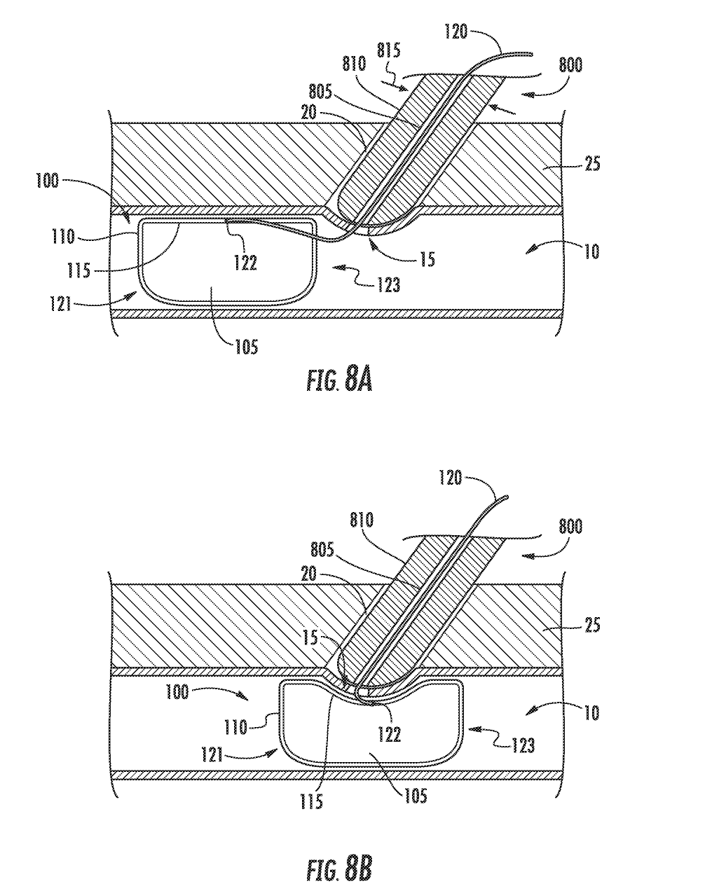

As discussed above, the various embodiments of a VCD described herein are configured to expand from a collapsed configuration into an expanded configuration within the vessel 10. Upon expanding, the VCD may apply a force to the wall of the vessel 10 such that the VCD is coupled to the vessel 10. FIGS. 6A and 6B illustrate the relationship between the size of the VCD in the expanded configuration and the resulting force applied to the vessel 10. In order to couple the expanded VCD to the wall of the vessel 10, the unrestricted diameter of the expanded VCD, specifically the expanded support frame in certain embodiments described above, preferably is at least similar to the diameter of the vessel 10 and preferably is larger. Because the diameter of the target vessel 10 may vary, it is an advantage that a single VCD will be useful for a range of vessel diameters. For example, the diameter of most human common femoral artery is between 6 mm and 10 mm. Of course, a given VCD will apply more force on a smaller diameter vessel than on a larger diameter vessel. This phenomena is illustrated in FIG. 6A, presenting the relationship between strain (resulting from the force applied on the vessel wall by VCD) and vessel diameter ranging between the maximal diameter (10 mm) and the minimal diameter (6 mm), for a given VCD. As is shown in this specific example, the strain on the minimal diameter vessel is higher by a factor of 8, as compared to the maximal diameter vessel.

On one hand, there is a minimal force (and resulting strain) required to assure appropriate coupling of the VCD to the wall of the vessel, and on the other hand, there is a maximal force (and resulting strain) that is considered safe because higher forces may injure the vessel. It therefore is desirable to minimize the ratio of the force applied on the vessel at the minimal diameter relative to the force applied at the maximal diameter. In order to decrease the difference between the forces applied on a smaller vessel and a larger one, it is now suggested to manufacture the VCD to have an unrestricted (free) diameter significantly larger than the working diameter range. An example of the effect of this novel approach is presented in FIG. 6B, which illustrates the relationship between strain ratio (the ratio of strain resulting from the force applied on a 6 mm vessel and a 10 mm vessel) and VCD free diameter. As is shown, the strain ratio is smaller for a VCD having a larger free diameter. For example, if the VCD is manufactured to have a free diameter of 12 mm, the strain ratio is more than 5; however, if the VCD free diameter is 20 mm, then the strain ratio drops to about 2.3. Therefore, in some embodiments of the VCD described above, the free diameter of the VCD is at least 10% larger, preferably at least 20% larger, and more preferably at least 40% larger, than the maximal working diameter. It is noted, however, that in some embodiments, increasing the VCD free diameter beyond a certain point may not be practical, as the strain in the VCD while being collapsed for delivery may exceed the elasticity range of the material used.

Vasculature Closure Systems and Methods of Use

FIG. 7A illustrates one embodiment of the VCD 100, similar to the embodiment illustrated in and described with reference to FIG. 1, in a collapsed configuration for delivery. Here, the VCD 100 is rolled into the collapsed configuration along the longitudinal axis of the VCD 100. Also shown with the VCD 100 is one embodiment of a containment mechanism 700, which may be part of a delivery system for deploying the VCD 100 into the vessel 10. The containment mechanism 700 is configured to releasably retain the VCD 100 in the collapsed configuration for delivery and positioning of the VCD 100 within the vessel 10 about the puncture site 15. Upon releasing the containment mechanism 700 (and consequent release of a compressive load holding the VCD 100 in the collapsed configuration) after suitable positioning within the lumen of the vessel 10, the VCD 100 is allowed to expand into its expanded configuration.

According to the embodiment shown in FIG. 7A, the containment mechanism 700 includes an implant holder 705 configured to extend from the distal end of a delivery device and at least one loop element 710. The loop element 710 encircles the VCD 100 to retain the VCD 100 in the collapsed configuration for delivery and positioning. The implant holder 705 is coupled to the loop element 710 and extends along the longitudinal axis of the VCD 100. As is shown in FIG. 7A, the loop element 710 includes a secured end 712 secured to the implant holder 705 and a looped end 714, optionally passing through a hole 716 defined in the implant holder 705, having a loop or other retaining means formed thereby. The loop element 710 may be formed of a flexible wire, as is shown. The containment mechanism also may include a retainer pin 718 configured for selective actuation during delivery of the VCD 100, which operates to release the looped end 714 from the retainer pin 718 and, thus, freeing the loop element 710 from around the VCD 100. As is shown, when in a secured configuration, the loop element 710 is positioned around and maintains the VCD 100 in the collapsed (e.g., rolled) configuration. The secured end 712 of the loop element 710 is secured by any suitable means to the implant holder 705, while the looped end 714 of the loop element 710 is releasably threaded over the retainer pin 718. The retainer pin 718 may be moveably secured to the implant holder 705 by any suitable means, such as, but not limited to, extending through one or more passages (as is shown in FIG. 7A), strapped thereto, passing through a channel, and the like, and extend proximally through a channel of the selected delivery device. In some embodiments, as is shown, the containment mechanism 700 includes a plurality of loop elements 710 encircling the VCD 100. The loop elements 710 may be formed from a single flexible wire or from multiple flexible wires.

In operation, the containment mechanism 700 releases the VCD 100 from the collapsed configuration by pulling the retainer pin 718 through the looped end 714 of the loop element 710. Any suitable actuating mechanism may be included with the chosen delivery device to allow pulling of the retainer pin 718. By pulling the retainer pin 718, the looped end 714 is released and the VCD 100 is freed and allowed to expand into the expanded configuration. Because the loop element 710 remains secured to the implant holder 705 at its secured end 712, the loop element 710 can be removed from the vessel 10 by removing the implant holder 705 and/or the delivery device utilized.

As discussed above, the containment mechanism 700 is configured for delivering and positioning the VCD 100 within the vessel 10 about the puncture site 15. In this manner, the containment mechanism 700 may be used to lead a proximal end 121 of the VCD 100 through the puncture site 15 and into the vessel 10. As is shown, the implant holder 705 includes a proximal element 720 configured to lead the proximal end 121 of the VCD 100 through the puncture site 15 and toward a proximal portion of the vessel 10. According to the embodiment of FIG. 7A, the proximal element 720 extends over and beyond the proximal end 121 of the VCD 100, creating an offset between the end of the proximal element 720 and the proximal end 121 of the VCD 100. In some embodiments, the offset is about 30 mm. In other embodiments, the offset is between about 20 mm and about 30 mm. In still other embodiments, the offset is about 10 mm or less, such as between about 1 mm and 8 mm.

According to one embodiment, the implant holder 705 is formed from a flexible film having a thickness between approximately 0.05 mm and approximately 5 mm, or between approximately 0.1 mm and approximately 0.5 mm in other embodiments, for example. The width of the implant holder 705 is between approximately 1 mm and approximately 5 mm in one embodiment, or between approximately 2 mm and approximately 4 mm in other embodiments, for example. The implant holder 705 may be made from any flexible, elastic, or super elastic material, including, but not limited to, biocompatible metallic materials (e.g., stainless steel, cobalt alloys, nickel-titanium or similar metals and alloys) biocompatible polymeric materials (e.g., PEEK, fluorocarbon polymers, polyamides, polyimides, polyethylenes, polypropylenes or similar polymers and copolymers), or any combination thereof. However, other suitable implant holder 705 configurations and dimensions may be provided, such as a more rigid member and/or one formed from different suitable materials, such as any other biocompatible material described herein.

An alternative embodiment of an implant holder 725 is shown in FIG. 7B. In this embodiment, a proximal element 727 of the implant holder 725 includes a concave portion 729. In this manner, the concave portion 729 may be configured to extend about the VCD 100. The remaining portions of the implant holder 725 may or may not also include a concave shape. In one embodiment, as is shown, the implant holder 725 has a concave shape extending along the full length of the implant holder 725. The concavity of the concave portion 729 or the full length of the implant holder 725 advantageously increases its rigidity, making it less prone to flexing while pulled against the vessel wall during delivery of the VCD 100. In some embodiments, the radius of curvature of the concave portion 729 or the full length of the implant holder 725 is from about 1 mm to about 10 mm, such as from about 2 mm to about 5 mm. In some embodiments, the radius of curvature is approximately the same as the radius of curvature of the VCD 100 in the collapsed configuration.

FIG. 7C illustrates one embodiment of a containment mechanism 750, similar to the containment mechanism 700 illustrated and described with reference to FIG. 7A, although certain differences in structure and function are described herein below. The containment mechanism 750 is configured to releasably retain the VCD 100 in the collapsed configuration for delivery and positioning of the VCD 100 within the vessel 10 about the puncture site 15. Upon releasing the containment mechanism 750 (and consequent release of a compressive load holding the VCD 100 in the collapsed configuration) after suitable positioning within the lumen of the vessel 10, the VCD 100 is allowed to expand into its expanded configuration. A particular difference between the containment mechanism embodiments shown in FIG. 7A and FIG. 7C is that the embodiment of the containment mechanism 750 shown in FIG. 7C includes an implant holder 755 including a distal element 772 configured to lead the distal end 123 of the VCD 100 toward a distal portion of the vessel 10.

According to the embodiment shown in FIG. 7C, the containment mechanism 750 includes the implant holder 755 configured to extend from the distal end of a delivery device and a loop element 760. The loop element 760 encircles the VCD 100 to retain the VCD 100 in the collapsed configuration for delivery and positioning. The implant holder 755 is coupled to the loop element 760 and extends along the longitudinal axis of the VCD 100. As is shown in FIG. 7C, the loop element 760 includes a secured end 762 secured to the implant holder 755 and a looped end 764, optionally passing through a hole 766 defined in the implant holder 755, having a loop or other retaining means formed thereby. The loop element 760 may be formed of a flexible wire, as is shown. The containment mechanism also may include a retainer pin 768 configured for selective actuation during delivery of the VCD 100, which operates to release the looped end 764 from the retainer pin 768 and, thus, freeing the loop element 760 from around the VCD 100. As is shown, when in secured configuration, the loop element 760 is positioned around and maintains the VCD 100 in the collapsed (e.g., rolled) configuration. The secured end 762 of the loop element 760 is secured by any suitable means to the implant holder 755, while the looped end 764 of the loop element 760 is releasably threaded over the retainer pin 768. The retainer pin 768 may be moveably secured to the implant holder 755 by any suitable means, such as, but not limited to, extending through one or more passages (as is shown in FIG. 7C), strapped thereto, passing through a channel, and the like, and extend proximally through a channel of the selected delivery device.

In operation, the containment mechanism 750 releases the VCD 100 from the collapsed configuration by pulling the retainer pin 768 through the looped end 764 of the loop element 760. Any suitable actuating mechanism may be included with the chosen delivery device to allow pulling of the retainer pin 768. By pulling the retainer pin 768, the looped end 764 is released and the VCD 100 is freed and allowed to expand into the expanded configuration. Because the loop element 760 remains secured to the implant holder 755 at its secured end 762, the loop element 760 can be removed from the vessel 10 by removing the implant holder 755 and/or the delivery device utilized.

According to one embodiment, the implant holder 755 is formed from a flexible film having a thickness between approximately 0.05 mm and approximately 5 mm, or between approximately 0.1 mm and approximately 0.5 mm in other embodiments, for example. The width of the implant holder 755 is between approximately 1 mm and approximately 5 mm in one embodiment, or between approximately 2 mm and approximately 4 mm in other embodiments, for example. The implant holder 755 may be made from any flexible, elastic, or super elastic material, including, but not limited to, biocompatible metallic materials (e.g., stainless steel, cobalt alloys, nickel-titanium or similar metals and alloys) biocompatible polymeric materials (e.g., PEEK, fluorocarbon polymers, polyamides, polyimides, polyethylenes, polypropylenes or similar polymers and copolymers), or any combination thereof. However, other suitable implant holder 755 configurations and dimensions may be provided, such as a more rigid member and/or one formed from different suitable materials, such as any other biocompatible material described herein.

As is shown in FIG. 7C, the implant holder 755 includes a proximal element 770 configured to lead the proximal end 121 of the VCD 100 through the puncture site 15 and toward a proximal portion of the vessel 10. In some embodiments, the proximal element 770 extends over and beyond the proximal end 121 of the VCD 100, creating an offset between the end of the proximal element 770 and the proximal end 121 of the VCD 100. In some embodiments, the offset is about 30 mm. In other embodiments, the offset is between about 20 mm and about 30 mm. In still other embodiments, the offset is about 10 mm or less, such as between about 1 mm and 8 mm. According to the embodiment of FIG. 7C, the implant holder 755 also includes a distal element 772 configured to lead the distal end 123 of the VCD 100 toward a distal portion of the vessel 10. In this manner, the distal element 772 may assist in detecting the puncture site 15 and crossing the puncture site 15 with the distal end 123 of the VCD 100 during pull back of the delivery system, which is a crucial step in properly delivering the VCD 100 within the vessel 10. In addition, the distal element 772 may be configured to increase the longitudinal rigidity of the collapsed VCD 100 and containment mechanism 750 combination, thereby advantageously enhancing the tactile feedback to the physician when pulling the combination against the wall of the vessel 10 around the puncture site 15.

The length of the distal element 772 may be configured to be the same as, longer, or shorter than the distal end 123 of the VCD 100. In one embodiment, the length of the distal element 772 is from about 0.5 mm to about 5 mm. In another embodiment, the length is from about 5 mm to about 10 mm. In still another embodiment, the length is from about 10 mm to about 20 mm. The distal element 772 may be made from the same material as the remainder of the implant holder 755, and in a preferred embodiment, the distal element 772 is cut from a single sheath of material. The distal element 772 may be manufactured by laser cutting, chemical etching, water-jet cutting, milling, and or using electrical arc processes. The manufacturing and/or shaping of the distal element 772 may be carried out in a separate process or in the same process as that used to make the proximal element 770 and other parts of the implant holder 755. Suitable processes include thermal setting, plastic deformation, casting, and/or any other means for shaping materials. The thickness of the distal element 772 may be the same as, thinner, or thicker than that of the proximal element 770 and other parts of implant holder 755. In a preferred embodiment, the thickness of the distal element 772 is from about 50 μm to about 150 μm. In another embodiment, the thickness is from about 10 μm to about 50 μm, or from about 150 μm to about 500 μm. In a preferred embodiment, the width of the distal element 772 is from about 2 mm to about 5 mm. In another embodiment, the width is from about 0.5 mm to about 3 mm, or from about 4 mm to about 8 mm.

Other embodiments of the implant holder are envisioned, which also have the advantageous features of the embodiments of the implant holders illustrated in and described with reference to FIGS. 7A and 7C. Some of these alternative embodiments are shown in FIGS. 7D-7F. FIG. 7D shows an embodiment of an implant holder 775 including a proximal element 777 and a distal element 779. According to this embodiment, the proximal element 777 and the distal element 779 are not integrally formed with each other. In other words, the proximal element 777 and the distal element 779 are formed separately from one another. The proximal element 777 and the distal element 779 may or may not be mechanically coupled to one another. In one embodiment, the proximal element 777 and the distal element 779 are uncoupled, such that the proximal element 777 and the distal element 779 are configured to move separately from one another. In this manner, the proximal element 777 and the distal element 779 may be configured to be removed separately from the VCD 100. For example, during removal of the containment mechanism, the distal element 779 may be removed first, followed at a later time by removal of the proximal element 777. This separate removal may allow for smoother removal of the containment mechanism as compared to simultaneous removal of the proximal element 777 and the distal element 779, which could impart relatively greater forces to the vessel 10 in the region about the puncture site 15.

FIG. 7E shows an embodiment of an implant holder 785 including a proximal element 787 and a distal element 789, and FIG. 7F shows an embodiment of an implant holder 795 including a proximal element 797 and a distal element 799. In each of these embodiments, the proximal element and the distal element are integrally formed with each other, and thus may be formed from a single piece of material. In addition, these embodiments of implant holders 785 and 795 are symmetric about a longitudinal axis of the implant holder.

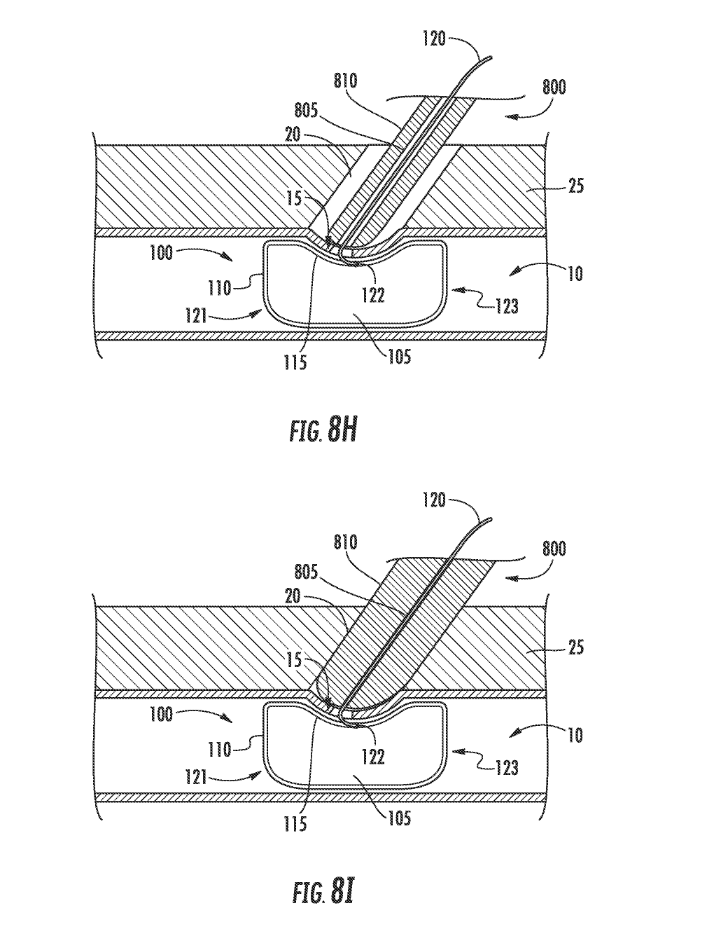

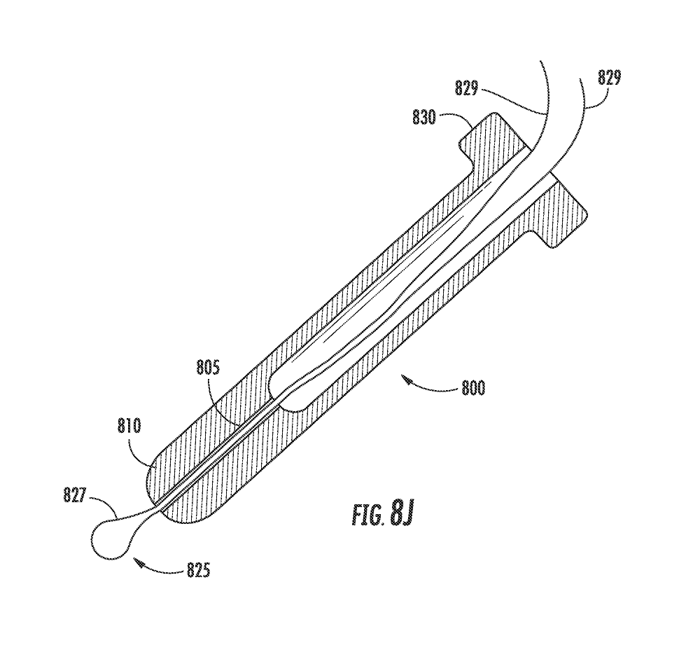

FIGS. 8A and 8B illustrate one embodiment of the VCD 100, similar to the embodiment illustrated in and described with reference to FIG. 1, in an expanded configuration within the vessel 10. Also shown with the VCD 100 is one embodiment of an inserting tool 800, which may be part of a delivery system for positioning the VCD 100 within the vessel 10. Although FIG. 8A shows the inserting tool 800 in use with the VCD 100, the inserting tool 800 may be used with any of the VCD embodiments described herein. When implanting one or more of the foregoing VCD embodiments to intravascularly seal the puncture site 15 in the vessel 10, the VCD should be positioned across the puncture site 15 such that the VCD covers both the distal and proximal sides of the puncture site 15. This “centering” of the VCD across the puncture site 15 may be a difficult process in some instances. For example, in some embodiments, centering is achieved or attempted to be achieved by pulling the tether of the VCD. However, as the VCD is expanded into the expanded configuration within the vessel 10, part of the VCD may become caught in the puncture site 15, preventing the VCD from crossing to the other side of the puncture site 15, or in some cases the VCD may inadvertently be pulled out of the vessel 10. The inserting tool 800 solves these problems by facilitating intravascular crossing of the puncture site 15 and thereby improving centering of the VCD.

According to the embodiment shown in FIGS. 8A and 8B, the inserting tool 800 is configured to intraluminally center the VCD 100 across the puncture site 15. The inserting tool 800 defines a lumen 805 extending through the inserting tool 800 and configured to receive the tether 120 of the VCD 100 threaded therethrough. In some embodiments, as is shown in FIGS. 8A and 8B, the inserting tool 800 includes a distal tip 810 configured to press against the wall of the vessel 10 without extending or penetrating through the puncture site 15.

In operation, the distal tip 810 of the inserting tool 800 is inserted through the access channel 20 formed in the tissue 25 by an introducer sheath and to the vessel 10 at the puncture site 15. The distal tip 810 is then gently pressed against the wall of the vessel 10, which allows an operator to readily feel the puncture site 15. In doing so, the distal tip 810 contacts the wall of the vessel 10 around the puncture site 15 without extending or penetrating through the puncture site 15, as is shown in FIG. 8A. The tether 120 is then pulled through the lumen 805 of the inserting tool 800, such that the distal end 123 of the expanded VCD 100 crosses the puncture site 15, while the distal tip 810 of the inserting tool 800 prevents the VCD 100 from penetrating the puncture site 15. In this manner, the VCD 100 is centered across the puncture site 15, as is shown in FIG. 8B. Although the VCD 100 is shown as crossing the puncture site 15 from a proximal portion of the vessel 10 to a distal portion of the vessel 10, the inserting tool 800 similarly may be used for a distal to proximal crossing of the puncture site 15.

The size and shape of the inserting tool 800 may vary and may be optimized, for example, depending on the sizes and shapes of the puncture site 15 and the access channel 20. In some embodiments, the outer diameter 815 of the distal tip 810 is from about 1 mm to about 10 mm. In one embodiment, the outer diameter 815 is from about 1.5 mm to about 7.5 mm. In another embodiment, the outer diameter 815 is from about 1 mm to about 2.5 mm. FIGS. 8C-8G illustrate example embodiments of the distal tip 810 of the inserting tool 800 having different shapes. Other shapes for the distal tip 810 are contemplated. In some embodiments, the distal tip 810 of the inserting tool 800 has an elongated cylindrical shape with an outer diameter 815 larger than the puncture site 15. For example, the outer diameter 815 of the distal tip 810 may be sized to be used with an 18 Fr introducer sheath, which may initially form a 7.5 mm puncture site 15. As the sheath is removed, the diameter of the puncture site 15 decreases as a result of contraction of the blood vessel muscles and thrombus formed. Accordingly, the distal end 815 having an outer diameter 815 of about 7 mm to about 7.5 mm should be able to penetrate the access channel 20 from the patient's skin to the vessel 10 and be stopped at the puncture site 15, as the wall of the vessel 10 around the puncture site 15 typically contracts faster than the tissue 25 around the access channel 20. At least part of inserting tool 800 may be formed of a radio-opaque material. In some embodiments, the distal tip 810 of inserting tool 800 is formed of a radio-opaque material, allowing fluoroscopic imaging of the distal tip 810 as it is inserted into the access channel 20 and pressed against the wall of the vessel 10.

As the puncture site 15 generally may be formed through the access channel 20 at an angle of about 30°-45° to the patient's skin, there may be an advantage to having (i) a distal tip 810 having a rounded surface, as is shown in FIG. 8C, (ii) a distal tip 810 having an angled and planar surface, as is shown in FIG. 8D, or (iii) a distal tip 810 having a combination of a rounded surface and an angled and planar surface, as is shown in FIG. 8E. A distal tip 810 having surfaces of other shapes and angles may be used for optimizing contact between the distal tip 810 of the inserting tool 800 and the puncture site 15 of the vessel 10. FIGS. 8F and 8G show embodiments of the distal tip 810 including a short protrusion 820 configured to extend into the puncture site 15 when the distal tip 810 of the inserting tool 800 is pressed against the against the wall of the vessel 10. The protrusion 820 may have a length of about 0.1 mm to about 6 mm and a diameter of 0.1 mm to about 8 mm. In some embodiments, the length of the protrusion 820 is from about 0.2 mm to about 4 mm and the diameter of protrusion 820 is from about 0.2 mm to about 5 mm. In still other embodiments, the length of the protrusion 820 is from about 0.2 mm to about 2 mm and the diameter of the protrusion 820 is from about 0.2 mm to about 3 mm.

FIGS. 8H and 8I show an embodiment of the inserting tool 800 including a distal tip 810 configured to reversibly expand from a collapsed configuration into an expanded configuration within the access channel 20. In this manner, the distal tip 810 is inserted into the access channel 20 while in the collapsed configuration, as is shown in FIG. 8H. When in the collapsed configuration, the outer diameter of the distal tip 810 is less than the inner diameter of the access channel 20, and the inner diameter of the lumen 805 is greater than the outer diameter of the tether 120. Upon expansion within the access channel 20, the outer diameter of the distal tip 810 increases to a size equal to or larger than the inner diameter of the access channel 20, such that the distal tip 810 seals against and grips the walls of the access channel 20, as is shown in FIG. 8I. Additionally, upon expansion within the access channel 20, the inner diameter of the lumen 805 decreases to a size equal to or less than the outer diameter of the tether 120, such that the lumen 805 seals against and grips the tether 120. In this manner, the expanded distal tip 810 functions in a like manner as the securing element 425 described above with reference to FIGS. 4A and 4B to maintain positioning of the VCD 100. Further, the expanded distal tip 810 promotes hemostasis at the puncture site 15 by sealing against the walls of the access channel 20 and the tether 120. After hemostasis is obtained, the distal tip 810 is collapsed for removal from the access channel 20.

As discussed above, the inserting tool 800 defines the lumen 805 extending through the inserting tool 800 and configured to receive the tether 120 of the VCD 100 threaded therethrough. In some embodiments, the inserting tool 800 is configured to ease threading of the tether 120 through the lumen 805, thereby simplifying use of the inserting tool 800. FIG. 8J illustrates an example embodiment of the inserting tool 800 including a means 825 for grasping the tether 120 and pulling the tether 120 through the lumen 805 of the inserting tool 800. In some embodiments, the grasping means 825 is pre-loaded in the lumen 805. In other embodiments, the grasping means 825 is loaded in the lumen 805 by an operator prior to or during the delivery procedure. In some embodiments, as is shown in FIG. 8J, the grasping means 825 includes a lasso loaded in the lumen 805. The lasso may include a loop 827 extending from the distal tip 810 of the inserting tool 800 and configured to receive the tether 120 of the VCD 100. The lasso also may include two proximal ends 829 extending from a proximal end 830 of the inserting tool 800.

In operation, the tether 120 of the VCD 100 is inserted through the loop 827 of the lasso. The proximal ends 829 of the lasso then are pulled such that the loop 827 is closed over the tether 120 and both the loop 827 and the tether 120 are pulled through the lumen 805 and out of the proximal end 830 of the inserting tool 800. At this stage, the tether 120 is separated from the lasso and may be used for positioning the VCD 100 within the vessel 10 as described herein. In another embodiment, one of the proximal ends 829 of the lasso is secured to the body of inserting tool 800, while the other proximal end 829 remains free for pulling. In this manner, the free proximal end 829 is pulled such that the loop 827 is closed over the tether 120 and both the loop 827 and the tether 120 are pulled through the lumen 805 and out of the proximal end 830 of the inserting tool 800. The grasping means 825 illustrated in FIG. 8J is merely one example embodiment of means for grasping the tether 120 and pulling the tether 120 through the lumen 805 of the inserting tool 800. Other embodiments of the grasping means 825 are contemplated. For example, the grasping means 825 may include a hook, knotting, snare clips, or other means known to those skilled in the art.

In some cases, during or after deployment of the VCD 100, it may be desirable to administer one or more therapeutic agents or other materials to the puncture site 15, for example to facilitate hemostasis at the external surface of the VCD 100 and/or the vessel 10 in proximity to the puncture site 15, or to the access channel 20 formed in the tissue 25 adjacent the vessel 10. Non-limiting examples of suitable therapeutic agents include hemostatic agents, vasodialators, vasoconstrictors, antimicrobial agents, and anesthetic agents, which are known in the art. In some embodiments, the inserting tool 800 may be configured and used to facilitate administration of such substances and materials. In one embodiment, the therapeutic agent includes a polysaccharide powder hemostatic agent as well known in the art.

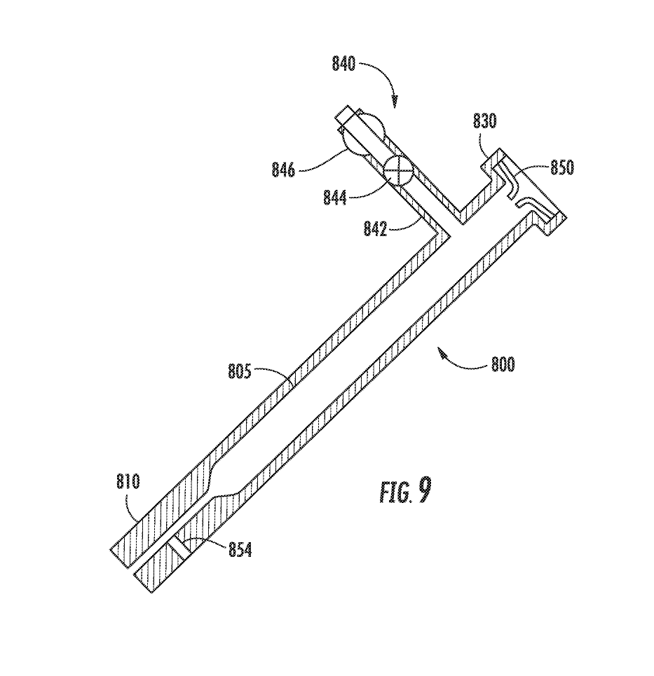

FIG. 9 illustrates an example embodiment of the inserting tool 800 configured to deliver therapeutic agents or other materials to the puncture site 15, the access channel 20, and/or the interface between the VCD 100 and the vessel 10. As is shown, the lumen 805 of the inserting tool 800 extends from the proximal end of the inserting tool 800 to the distal tip 810 of the inserting tool 800. In this manner, the lumen 805 is configured for delivering therapeutic agents or other materials therethrough. The inserting tool 800 includes means 840 for delivering the therapeutic agents or other materials into the lumen 805. In some embodiments, as is shown in FIG. 9, the delivery means 825 includes a tube 842 extending from and in fluid communication with the lumen 805. The delivery means 825 also may include a valve 844 positioned on the tube 842 and configured to control delivery of the therapeutic agents or other materials into the lumen 805. The delivery means 825 also may include a luer lock 846 positioned on a distal end of the tube 842 and configured to connect a syringe or bellows to the inserting tool 800. In some embodiments, the proximal end 830 of the inserting tool 800 is configured for direct delivery of therapeutic agents or other materials into the lumen 805. In such embodiments, the inserting tool 800 may include a sealing means, such as a valve 850, positioned at the proximal end 830 and configured to control delivery of the therapeutic agents or other materials into the lumen 805. The valve 850 may be a hemostatic valve configured to prevent or reduce blood flow therethrough. In some embodiments, the inserting tool 800 defines a side hole 854 extending through a sidewall of the inserting tool 800 and in fluid communication with the lumen 805. As is shown in FIG. 9, the side hole 854 is positioned near the distal end of the inserting tool 800.

In one embodiment, inserting tool 800 is used to deliver a clotting promoting agent such as thrombin, protamine, or other thrombus promoting factors or agents, such as zeolite powder or chitosan granule. In another embodiment, the inserting tool 800 is used to deliver drugs, factors, or agents that may induce vasodilatation, such as nitroglycerin or Papaverine and similar agents, or vasoconstriction agents, antibiotic or antibacterial drugs, glue, such as cyanoacrylate or biological adhesive, collagen or any other material, agent, factor, or drug that may improve sealing, promote healing, or decrease bleeding at the puncture site 15, the access channel 20, and/or the interface between the VCD 100 and the vessel 10.

In one embodiment, after the VCD 100 is delivered, a hemostasis promoting drug, factor or agent is applied directly to the surface of the patient's body in proximity to the puncture site 15 for expediting complete full hemostasis. In another embodiment, a hemostatic dressing or plaster or bandage is used over the puncture site 15, e.g., Quikclot™, Celox™ or HemCon™. In another embodiment, the hemostatic dressing is also used to secure the tether 120 of the VCD 100.

It is appreciated that these and many other advantages will be appreciated, and modifications and variations of the devices, systems, and methods described herein, such as dimensional, size, and/or shape variations, will be apparent to those skilled in the art from the foregoing detailed description. Such modifications and variations are intended to come within the scope of the appended claims.

We claim:

1. A vasculature closure device, comprising:
an expandable support frame deployable within a vessel;
a sealing membrane at least partially supported by the support frame; and
a cross-member support extending across at least a portion of the sealing membrane;
wherein, upon expanding the support frame, the device is configured to intraluminally position the sealing membrane against a puncture site existing in a wall of the vessel;
wherein the cross-member support comprises a flexible wire configured to maintain the sealing membrane against the puncture site; and
wherein the flexible wire comprises a first wire segment and a second wire segment defining an X-shape of the cross-member support and configured to distribute forces applied to the cross-member support.

2. The vasculature closure device of claim 1, wherein the flexible wire is attached to opposite sides of the support frame.

3. The vasculature closure device of claim 1, wherein the flexible wire is formed of a suture material.

4. The vasculature closure device of claim 1, wherein the flexible wire extends over the sealing membrane and is configured to be positioned between the sealing membrane and the wall of the vessel.

5. The vasculature closure device of claim 1, wherein the flexible wire extends beneath the sealing membrane and is configured to be positioned between the sealing membrane and a flow of blood through the vessel.

6. The vasculature closure device of claim 1, further comprising a tether attached to the flexible wire.

7. The vasculature closure device of claim 6, wherein the tether is attached to a center of the X-shape of the cross-member support, and wherein the first wire segment and the second wire segment are configured to distribute pulling forces applied to the cross-member support via the tether to reduce bending of the support frame.

8. The vasculature closure device of claim 1, wherein an intermediate portion of the flexible wire is coupled to the sealing membrane by a coupler attached to the sealing membrane and extending over the intermediate portion of the flexible wire.

9. The vasculature closure device of claim 8, wherein the intermediate portion of the flexible wire is movable between the coupler and the sealing membrane.

10. The vasculature closure device of claim 8, wherein the coupler is positioned at about a center of a longitudinal axis of the cross-member support.

11. The vasculature closure device of claim 8, wherein the coupler comprises a wire.

12. The vasculature closure device of claim 8, wherein the coupler comprises a patch.

13. The vasculature closure device of claim 8, wherein the coupler is formed of a biodegradable material.

14. The vasculature closure device of claim 1, wherein the first wire segment extends between opposite sides of the support frame and the second wire segment extends between opposite sides of the support frame.

15. The vasculature closure device of claim 1, wherein an intermediate portion of the flexible wire is coupled to the sealing membrane.

16. The vasculature closure device of claim 1, wherein the device is configured for rolling and unrolling along a longitudinal axis generally aligned with and extending along the length of the vessel.

17. The vasculature closure device of claim 1, wherein the support frame is configured to expand into an expanded configuration having a radius of curvature greater than a radius of curvature of the vessel.

18. The vasculature closure device of claim 1, wherein the support frame is formed of a pre-shaped material configured to expand from a collapsed configuration into a pre-shaped expanded configuration.

19. The vasculature closure device of claim 18, wherein the pre-shaped material comprises at least one of a shape memory metal and a shape memory polymer.

20. The vasculature closure device of claim 1, wherein the sealing membrane defines an outer edge about its periphery and further comprises a plurality of tabs extending about the outer edge, and wherein the sealing membrane is attached to the support frame by the plurality of tabs.

21. The vasculature closure device of claim 20, wherein each of the tabs wraps around a portion of the support frame and is bonded to the sealing membrane.

22. The vasculature closure device of claim 1, wherein the support frame defines a plurality of holes, and wherein the sealing membrane is attached to the support frame by a plurality of anchors extending through the plurality of holes.

23. The vasculature closure device of claim 22, wherein the anchors define a stud-like shape and are formed of a glue or an adhesive.

24. The vasculature closure device of claim 1, wherein the support frame is embedded within the sealing membrane.

25. The vasculature closure device of claim 1, wherein the support frame comprises a first wing, a second wing positioned opposite the first wing, and at least one tab extending from the first wing and configured to increase a longitudinal stiffness of the device in a collapsed configuration, wherein the at least one tab is configured to contact the support frame at or near a centerline of the support frame when the device is in the collapsed configuration, and wherein the at least one tab is configured to prevent the first wing from applying pressure on the sealing membrane when the device is in the collapsed configuration.

26. The vasculature closure device of claim 25, wherein the device is configured for rolling into the collapsed configuration and unrolling into an expanded configuration, wherein the second wing is rolled over the first wing and the at least one tab when the device is in the collapsed configuration, and wherein the at least one tab is configured to apply a force to the support frame for unrolling into the expanded configuration.

27. The vasculature closure device of claim 1, further comprising a tether extending away from the sealing membrane, and a securing element coupled to the tether, wherein the securing element is configured to engage an access channel formed in a tissue adjacent the puncture site to prevent intraluminal migration of the device.

28. A vasculature closure device, comprising:
an expandable support frame deployable within a vessel;
a sealing membrane at least partially supported by the support frame;
a tether extending away from the sealing membrane; and
a securing element coupled to the tether;
wherein, upon expanding the support frame, the device is configured to intraluminally position the sealing membrane against a puncture site existing in a wall of the vessel; and
wherein the securing element is configured to engage an access channel formed in a tissue adjacent the puncture site to prevent intraluminal migration of the device.

29. The vasculature closure device of claim 28, wherein the securing element is configured to expand from a collapsed configuration for delivery into the access channel into an expanded configuration for engaging the access channel.

30. The vasculature closure device of claim 28, wherein the securing element comprises a pin configured to rotate from an upright position to an angled position for engaging the access channel.
